(19) United States
(12) Patent Application Publication
(10) Pub. No.: US 2012/0100108 A1
(43) Pub. Date: Apr. 26, 2012

(54) COMPOSITIONS AND METHODS OF TREATING NO-OPTION CRITICAL LIMB ISCHEMIA (CLI)

(75) Inventors: Ronnda Bartel, Ann Arbor, MI (US); Sharon Watling, Ann Arbor, MI (US)

(73) Assignee: Aastrom Biosciences, Inc., Ann Arbor, MI (US)

(21) Appl. No.: 13/158,298

(22) Filed: Jun. 10, 2011

Related U.S. Application Data
(60) Provisional application No. 61/353,512, filed on Jun. 10, 2010.

(51) Int. Cl. A61K 35/12; A61P 43/00

(52) U.S. Cl. 424/93.3

(57) ABSTRACT

The present invention provides methods for treating critical limb ischemia (CLI), including increasing wound healing, decreasing wound size, increasing survival-free amputation, preventing amputation, preventing or delaying de novo gangrene, increasing survival probability, and preventing or delaying death, in subjects who prevent a vascular occlusion that cannot be resolved by using a standard method of revascularization, i.e. a subject with “no-option” CLI. Methods of the invention include administering to a subject with no-option CLI an isolated cell composition for tissue repair comprising a mixed population of cells of hematopoietic, mesenchymal and endothelial lineage, wherein the viability of said cells is at least 80% and the composition contains: a) about 5-75% viable CD90⁺ cells with the remaining cells in said composition being CD45⁺; b) less than 2 μg/ml of bovine serum albumin; c) less than 1 μg/ml of a enzymatically active harvest reagent; and d) substantially free of mycoplasma, endotoxin, and microbial contamination.

### RELATED APPLICATIONS

This application claims the benefit of U.S. Ser. No., 61/353,512 filed Jun. 10, 2010, the contents of which are incorporated by reference in its entirety.

### FIELD OF THE INVENTION

The present invention relates to compositions of mixed cell populations, their subsequent use in vivo for tissue repair and processes, and, in particular, to the treatment of critical limb ischemia (CLI) for those patients and subjects who present a vascular occlusion that cannot be resolved by using a standard method revascularization.

### BACKGROUND OF THE INVENTION

Regenerative medicine harnesses, in a clinically targeted manner, the ability of regenerative cells, e.g., stem cells and/or progenitor cells (i.e., the unspecialized master cells of the body), to renew themselves indefinitely and develop into mature specialized cells. Stem cells are found in embryos during early stages of development, in fetal tissue and in some adult organs and tissue. Embryonic stem cells (hereinafter referred to as “ESCs”) are known to become many if not all of the cell and tissue types of the body. ESCs not only contain all the genetic information of the individual but also contain the nascent capacity to become any of the 200+ cells and tissues of the body. Thus, these cells have tremendous potential for regenerative medicine. For example, ESCs can be grown into specific tissues such as heart, lung or kidney which could then be used to repair damaged and diseased organs. However, ESC derived tissues have clinical limitations. Since ESCs are necessarily derived from another individual, i.e., an embryo, there is a risk that the recipient's immune system will reject the new biological material. Although immunosuppressive drugs to prevent such rejection are available, such drugs are also known to block desirable immune responses such as those against bacterial infections and viruses.

Moreover, the ethical debate over the source of ESCs, i.e., embryos, is well-chronicled and presents an additional and, perhaps, insurmountable obstacle for the foreseeable future.

Adult stem cells (hereinafter interchangeably referred to as “ASCs”) represent an alternative to the use of ESCs. ASCs reside quietly in many non-embryonic tissues, presumably waiting to respond to trauma or other destructive disease processes so that they can heal the injured tissue. Notably, emerging scientific evidence indicates that each individual carries a pool of ASCs that may share with ESCs the ability to become many if not all types of cells and tissues. Thus, ASCs, like ESCs, have tremendous potential for clinical applications of regenerative medicine.

ASC populations have been shown to be present in one or more of bone marrow, skin, muscle, liver and brain. However, the frequency of ASCs in these tissues is low. For example, mesenchymal stem cell frequency in bone marrow is estimated at between 1 in 100,000 and 1 in 1,000,000 nucleated cells Thus, any proposed clinical application of ASCs from such tissues requires increasing cell number, purity, and maturity by processes of cell purification and cell culture.

Although cell culture steps may provide increased cell number, purity, and maturity, they do so at a cost. This cost can include one or more of the following technical difficulties: loss of cell function due to cell aging, loss of potentially useful cell populations, delays in potential application of cells to patients, increased monetary cost, increased risk of contamination of cells with environmental microorganisms during culture, and the need for further post-culture processing to deplete culture materials contained with the harvested cells.

More specifically, all final cell products must conform with rigid requirements imposed by the Federal Drug Administration (FDA). The FDA requires that all final cell products must minimize “extraneous” proteins known to be capable of producing allergenic effects in human subjects as well as minimize contamination risks. Moreover, the FDA expects a minimum cell viability of 70%, and any process should consistently exceed this minimum requirement.

While there are existing methods and apparatus for separating cells from unwanted dissolved culture components and a variety of apparatus currently in clinical use, such methods and apparatus suffers from a significant problem—cellular damage caused by mechanical forces applied during the separation process, exhibited, for instance, by a reduction in viability and biological function of the cells and an increase in free cellular DNA and debris. Furthermore, significant loss of cells can occur due to the inability to both transfer all the cells into the separation apparatus as well as extract all the cells from the apparatus. In addition, for mixed cell populations, these methods and apparatus can cause a shift in cell profile due to the preferential loss of larger, more fragile subpopulations.

Thus, there is a need in the field of cell therapy, such as tissue repair, tissue regeneration, and tissue engineering, for cell compositions that are ready for direct patient administration with substantially high viability and functionality, and with substantial depletion of materials that were required for culture and harvest of the cells. Furthermore, there are needs for reliable processes and devices to enable production of these compositions that are suitable for clinical implementation and large-scale commercialization of these compositions as cell therapy products.

### SUMMARY OF THE INVENTION

The invention provides a method of treating critical limb ischemia (CLI) in a subject, wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination, thereby improving or preventing the clinical consequence of critical limb ischemia (CLI). The isolated cell composition for tissue repair is also referred to herein as the tissue repair cell (TRC) composition. The formulation of this composition used in the clinical trials described in Examples 1 and 2 is also known as ixmyelocel-T.

In certain embodiments of this method, the standard method of revascularization is an open surgical procedure or a percutaneous endovascular procedure. Furthermore, the presence of a vascular occlusion that cannot be resolved by using a standard method of revascularization may be determined by physical examination, angiographic imaging, color flow duplex ultrasound, or any combination thereof.

According to certain aspects of this method, the subject may present a vascular occlusion in a lower extremity. Alternatively, or in addition, the subject may present recurring ischemic rest pain for at least 2 weeks, ulceration, or gangrene with absent pulses in an extremity. When a subject presents a vascular occlusion in a lower extremity, the subject may further present recurring ischemic rest pain for at least 2 weeks, ulceration, or gangrene in the foot or toe with absent pedal pulses, and with either a toe systolic pressure of equal to or less than 50 mm Hg or ankle systolic pressure of equal to or less than 70 mm Hg.

Exemplary clinical consequences of no-option critical limb ischemia (CLI) include, but are not limited to, increased rest pain, decreased mobility of a limb (arm or leg), ulceration, increased wound size (doubling of wound size), decreased or impaired wound healing, de novo gangrene, decreased or absent pulse at extremity, tissue loss (tissue necrosis), amputation (for instance, of a digit, such as a finger or toe, which would not constitute a major amputation), major amputation (defined as, for example, an amputation at or above the talus on the leg), or death. Decreased function of an affected limb includes, but is not limited to, decreased range of motion, decreased strength, or decreased endurance for physical exertion of the limb. In certain aspects of this method, the limb is a leg and a decreased function of an affected limb includes decreased walking distance or decreased walking time.

The treatment of the subject presenting a vascular occlusion that cannot be resolved by using a standard method of revascularization achieves a clinical goal. Exemplary clinical goals include, but are not limited to, decreased pain, increased function of an affected limb, decreased wound size, increased wound healing, delay or prevention of de novo gangrene, delay or prevention of amputation, or increased survival.

When the clinical goal is decreased pain, decreased pain is determined by comparing a demand from the subject for administration of a pain medicine or a dosage of a pain medication from a time period prior to administration of the composition to a demand from the subject for administration of a pain medicine or a dosage of a pain medication from a time point following administration of the composition, wherein a decreased demand or a decreased dosage indicates that the treatment decreased the pain of the subject following administration of the composition.

When the clinical goal is increased function of an affected limb, increased function of an affected limb is determined by comparing a range of motion, a strength, or an endurance measurement for physical exertion of the limb from a time period prior to administration of the composition to a range of motion, a strength, or an endurance measurement for physical exertion of the limb from a time point following administration of the composition, wherein an increased range of motion, increased strength, or increased endurance measurement indicates that the treatment increased the function of the affected limb of the subject following administration of the composition.

When the clinical goal is decreased wound size, decreased wound size is determined by comparing an area, circumference, or depth measurement of a wound from a time period prior to administration of the composition to an area, circumference, or depth measurement of a wound from a time point following administration of the composition, wherein a decreased area, circumference, or depth measurement indicates that the treatment decreased size of a wound following administration of the composition.

When the clinical goal is increased wound healing, increased wound healing is determined by comparing a measurement of active inflammation, angiogenesis, collagen disposition, fibroplasia, granulation tissue formation, epithelialization, contraction, or remodeling of a wound from a time period prior to administration of the composition to a measurement of active inflammation, angiogenesis, collagen disposition, fibroplasias, granulation tissue formation, epithelialization, contraction, or remodeling of a wound from a time point following administration of the composition, wherein an increased measurement of active inflammation, angiogenesis, collagen disposition, fibroplasia, granulation tissue formation, epithelialization, contraction, or remodeling indicates that the treatment increased wound healing following administration of the composition.

When the clinical goal is delay or prevention of de novo gangrene, delay or prevention of de novo gangrene is determined by comparing a measurement of tissue necrosis from a time period prior to administration of the composition to a measurement of tissue necrosis from a time point following administration of the composition, wherein an identical or decreased measurement of tissue necrosis indicates that the treatment delayed or prevented the formation of de novo gangrene following administration of the composition.

When the clinical goal is delay or prevention of amputation, delay or prevention of amputation is determined by comparing the prognosis for amputation in the subject from a time period prior to administration of the composition to the prognosis for either amputation in the subject following administration of the composition, wherein an increase in the time required until amputation or a cancellation of the amputation procedure due to recovery indicates that the treatment delayed or prevented the amputation of the affected limb, respectively.

When the clinical goal is increased survival, increased survival is determined by comparing the prognosis for survival in the subject from a time period prior to administration of the composition to the prognosis for survival in the subject following administration of the composition, wherein an increase in predicted survival time indicates that the treatment increased survival of the subject following administration of the composition.

The composition may be administered by intramuscular injection at one or more sites. In a preferred embodiment, the composition is injected at 20 sites.

The cells of the composition are derived from mononuclear cells. These mononuclear cells are derived from bone marrow, peripheral blood, umbilical cord blood or fetal liver.

Optionally, cells of the composition are in a pharmaceutical-grade electrolyte solution suitable for human administration. In certain aspects, the composition is substantially free of horse serum and/or fetal bovine serum.

In certain aspects of the invention, at least 10% of the CD90⁺ cells of the composition co-express CD15. Alternatively, or in addition, the CD45⁺ cells of the composition are CD14⁺, CD34⁺ or VEGFR1⁺.

In certain embodiments of this method, the total number of viable cells in the composition is 35 million to 300 million. In a preferred embodiment, the composition contains an average of between 90-180±10⁶ viable cells. Moreover, the cells may be in a volume less than 15 milliliters, 10 milliliters, or 7.5 milliliters.

The invention also provides a method of increasing amputation-free survival in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45^{±}; b) less than 2 μg/ml of bovine serum albumin; c) less than 1 μg/ml of a enzymatically active harvest reagent; and d) substantially free of mycoplasma, endotoxin, and microbial contamination. In certain embodiments of this method, the amputation-free survival is increased in the treated subject when compared to an untreated subject, wherein the untreated subject is also diagnosed with critical limb ischemia (CLI) and also presents a vascular occlusion that cannot be resolved by using a standard method of revascularization. Amputation-free survival is defined as the time of administration of the composition until an amputation is performed, the subject dies, or the combination occurs.

The invention provides a method of preventing major amputation in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. In certain embodiments of this method, the vascular occlusion occurs in a leg. When the vascular occlusion occurs in a leg, the major amputation is an amputation at or above the talus on the leg. In certain aspects of this method, a major amputation is prevented from the time of administration of the composition until 1, 2, 3, 4, 5, 6, 7, 8, 9, 10, 15, 20, or 25 years after administration. Alternatively, or in addition, a major amputation is prevented because the extremity is revascularized, as confirm, for instance, by physical examination, angiographic imaging, color flow duplex ultrasound, or any combination thereof. A subject who experiences revascularization in combination with function of the extremity will avoid major amputation indefinitely, and, therefore, the major amputation has been prevented.

The invention provides a method of delaying the onset of de novo gangrene, tissue loss, amputation, or death in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. In certain embodiments of this method, the onset of de novo gangrene, tissue loss, amputation, or death is delayed in the treated subject when compared to an untreated subject, wherein the untreated subject is also diagnosed with critical limb ischemia (CLI) and also presents a vascular occlusion that cannot be resolved by using a standard method of revascularization. In certain aspects of this method, amputation includes minor and major amputation.

A subject with no-option CLI, who also has an underlying medical condition like morbid obesity, advanced diabetes, or advanced age (with poor general health), may not be able to avoid the more severe consequences of no-option CLI forever, however, they may benefit from these methods by delaying the onset of these events for a sufficient time to experience a significant increased in quality of life. Furthermore, an elderly patient may benefit by avoiding amputation until morbidity arises from age rather than no-option CLI, thereby, benefitting by an increased quality of life for the interim. Therefore, because the subject may already be in poor health, independent of his or her affliction with no-option CLI, the concept of “treating” no-option CLI includes improving mobility, decreasing pain, improving wound healing, decreasing wound size, and delaying tissue loss, amputation, and death. Although the treatment for no-option CLI could be a cure in an otherwise healthy individual, the measure of success for treating a subject with no-option CLI in the average subject includes ameliorating an existing symptom or delaying the onset of a worse symptom.

The invention provides a method of increasing survival probability in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. In certain embodiments of this method, the survival probability is increased in the treated subject when compared to an untreated subject, wherein the untreated subject is also diagnosed with critical limb ischemia (CLI) and also presents a vascular occlusion that cannot be resolved by using a standard method of revascularization. Survival probability is another method of expressing the time to treatment failure, or the likelihood that the treatment will be successful. The data provided herein demonstrate that individuals with no-option CLI experience a statistically significant benefit from administration of this composition because they survive for a longer time before experiencing a no-option CLI-induced adverse event.

In certain embodiments of the methods provided herein, the composition is administered to a subject who presents a vascular occlusion that cannot be resolved by using a standard method of revascularization, in combination with another therapy. For instance, if the subject suffers from an underlying atherosclerosis in the limb undergoing treatment, or in another part of his or her body, the composition is administered in combination with a pharmaceutical agent. Contemplated pharmaceutical agents reduce lipids (lipid or cholesterol reduction therapy), reduce platelet aggregation or platelet attachment to the walls of the vasculature (anti-platelet therapy), or reduce blood pressure (anti-hypertensive therapy). Moreover, the subject of the present methods may have a wound associated with no-option CLI on the treated limb, or on another part of his or her body. Thus, the composition is administered in combination with topical or systemic wound care. Exemplary wound care includes, but is not limited to, pharmaceutical agents to decrease infection (like antibiotics), decrease inflammation, promote healing (antioxidants), and promote vascularization (pro-angiogenic factors); matrices or scaffolds to provide a substrate upon which to grow tissue for the wound; and surgical intervention to removal of dead, damaged, or infected tissue (debridement).

Patients/subjects who develop no-option CLI are often obese, diabetic, and/or elderly. Moreover, these patients experience heart disease at a higher rate than the general population. Thus, the methods provided herein may also be used in combination with treatments for obesity (for instance, drugs including olitstat and the non-prescription version, alli), heart disease (for instance, drugs used to combat high cholesterol or high blood pressure), and diabetes (for example, insulin for type I and weight-loss therapy for type II).

In one aspect the invention provides an isolated cell composition for tissue repair containing a mixed population of cells. The cells are in a pharmaceutical-grade electrolyte solution suitable for human administration. The cells are derived from mononuclear cells. For example, the cells are derived from bone marrow, peripheral blood, umbilical cord blood or fetal liver. The cells are of hematopoietic, mesenchymal and endothelial lineage. The viability of cells is at least 80%, 85%, 90%, 95%, 96%, 97%, 98%, 99% or greater. The total number of viable cells in the composition is 35 million to 300 million and in volume less than 25 ml, 20 ml, 15 ml, 10 ml, 7.5 ml, 5 ml or less. At least 5% of the viable cells in the composition are CD90⁺. For example, 10%, 20%, 30%, 40%, 50%, 60%, 70%, 75% or more are CD90⁺. In some as at least 5%, 10%, 15%, 20%, 50% or more of the CD90⁺ co-express CD15. Preferably, the cells are about 5-75% viable CD90⁺ with the remaining cells in the composition being CD45⁺. The CD45⁺ cells are CD14⁺, CD34⁺ or VEGFR1⁺.

The composition is substantially free of components used during the production of the cell composition, e.g., cell culture components such as bovine serum albumin, horse serum, fetal bovine serum, enzymatically active harvest reagent (e.g., trypsin) and substantially free of mycoplasma, endotoxin, and microbial contamination . Preferably, the composition contain 10, 5, 4, 3, 2, 1, 0.1, 0.05 or less μg/ml bovine serum albumin and 5, 4, 3, 2, 1, 0.1, 0.05 μg/ml enzymatically active harvest reagent.

This composition and methods of making this composition are provided in International Application No. PCT/US2007/023302, Publication No. WO 2008/054825, the contents of which are incorporated herein in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

Various other objects, features and attendant advantages of the present invention will be more fully appreciated as the same becomes better understood from the following detailed description when considered in connection with the accompanying drawings in which like reference characters designate like or corresponding parts throughout the several views and wherein:

FIG. 1A-D is a series of photographs depicting the process of expanding bone marrow cells in a bioreactor following harvest.

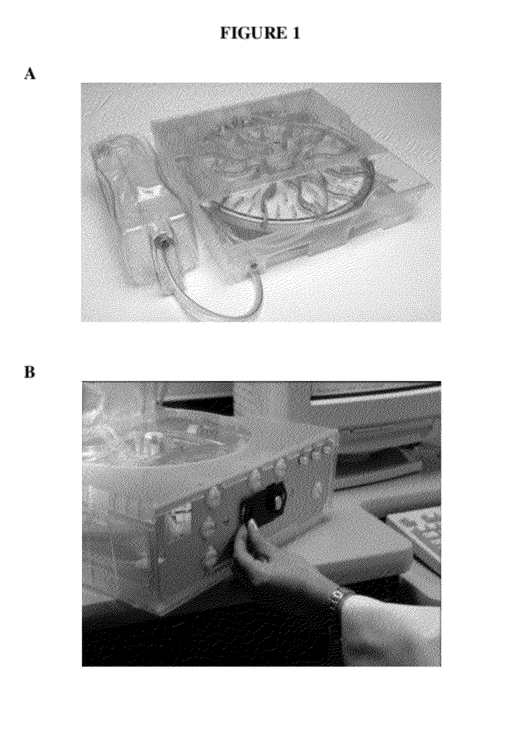

FIG. 2A-B is a pair of graphs depicting the frequency distribution of cell types found in starting bone marrow (A) versus TRC populations following expansion. TRC expansion according methods of the invention also increases the number of early stage cells found in bone marrow. The differences between A and B demonstrate that the frequency of cell types shifts towards stem and progenitor cells following expansion of bone marrow cells into TRC populations in the bioreactor.

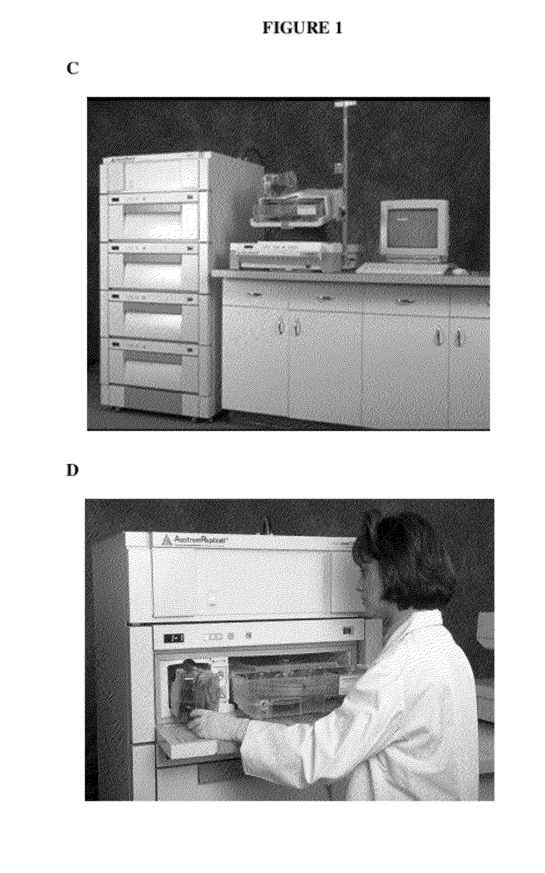

FIG. 3 is a graph depicting a phenotypic analysis of bone marrow aspirate and TRC product samples. Flow cytometric analysis was performed on aspirate samples before automated single-pass perfusion culture and on the TRC products. Patients that had samples with complete phenotypic analyses for both aspirate and TRC samples were included (n=19). Figures above the bars indicate the average number of cells in millions.

FIG. 4 is a graph depicting a Kaplan-Meier survival plot of time to first occurrence of treatment failure, a composite endpoint of major amputation, doubling of wound total surface area, occurrence of de novo gangrene or death. Censored observations are indicated by “+” symbols.

FIG. 5A-B is a pair of graphs depicting a Kaplan-Meier survival plot of amputation-free survival at the first interim analysis timepoint (A) and the final study database lock (B), respectively. Censored observations are indicated by “+” symbols.

FIG. 6 is a graph depicting the results of the first interim analysis for amputation rates over a period of 6 months (expressed as the percent of patients amputated) over time (the duration of the 6-month window) in either the control (placebo-treated) group versus TRC-treated group. Both treatment conditions were provided to subjects diagnosed with no-option critical limb ischemia. The data from the first interim analysis demonstrate that 19% of TRC-treated population experienced amputation, whereas 43% of the control group were amputated (p=0.14, Fischer' Exact Test).

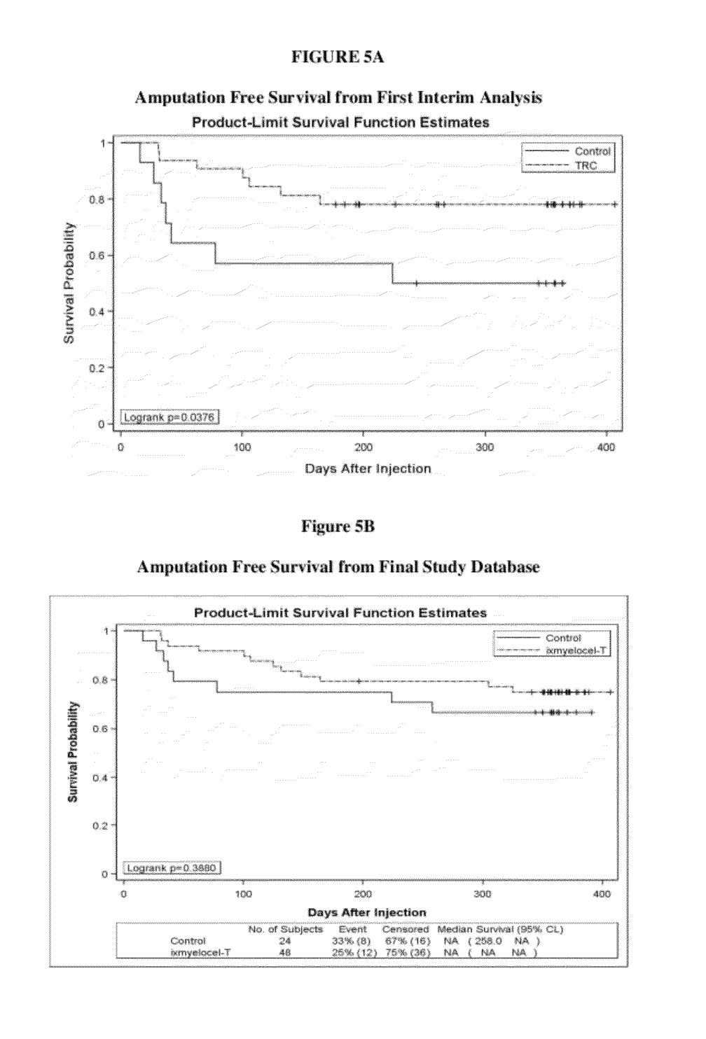

FIG. 7 is a graph depicting the data from the first interim analysis for major amputation rates over a period of 12 months (expressed as the percent of patients amputated) over time (data gathered and shown at 6- and 12-month time points) in either the control (placebo-treated) group versus TRC-treated group. Both treatment conditions were provided to subjects diagnosed with no-option critical limb ischemia. At the first interim analysis, at both 6- and 12-months, the data demonstrate that 18% of TRC-treated population experienced amputation, whereas 36% of the control group were amputated (p=0.39, Fischer' Exact Test).

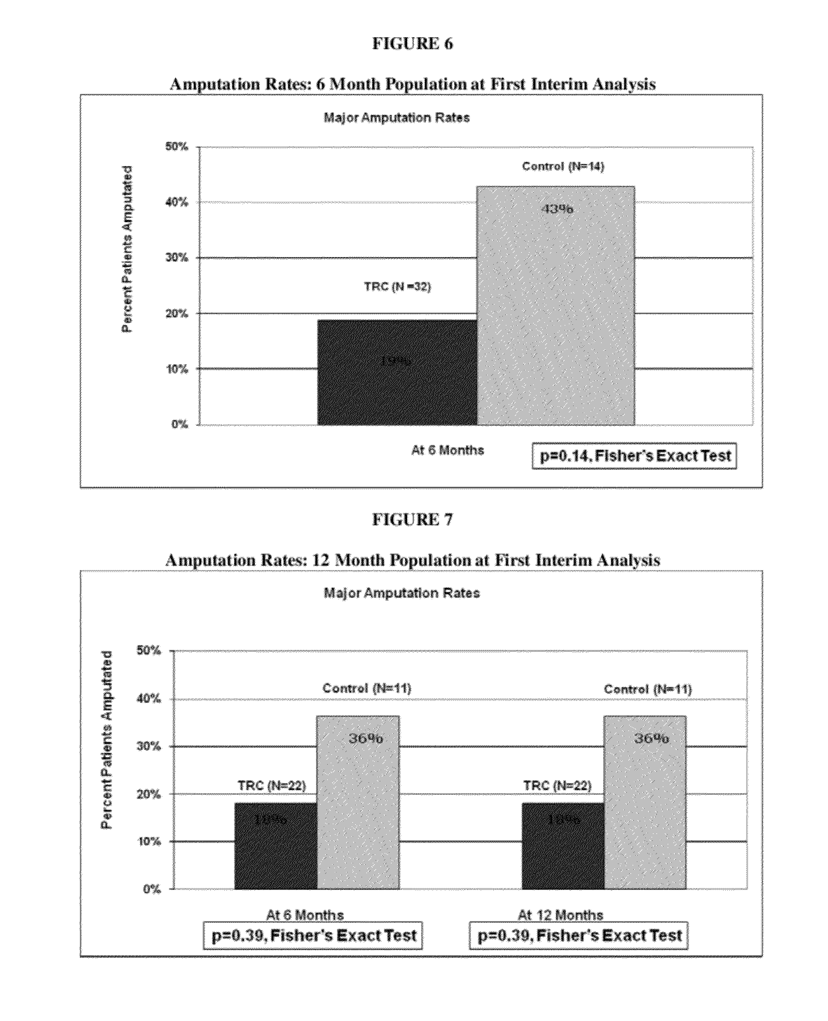

FIG. 8 is a graph depicting the data from the first interim analysis for complete wound healing rate in patients completing 12-months of follow up at 6 and 12-month time points. There was no statistical difference between control and TRC-patient groups at the 6-month time point (P=1.00, Fisher's exact test). At the first interim analysis, at the 12-month time point, the greater incidence of wound healing in the TRC-treated group was also not statistically significant, due to the small sample size (P=0.61, Fisher's exact test).

FIG. 9 is a graph depicting a Kaplan-Meier survival plot of amputation-free survival (AFS) in patients with wounds at baseline, at the final study database lock.

FIG. 10 is a graph depicting a Kaplan-Meier survival plot of time to treatment failure (TTF) in patients with wounds at baseline, at the final study database lock.

### DETAILED DESCRIPTION OF THE INVENTION

The present invention is based on the discovery of compositions and methods of producing cells for cell therapy. The compositions are a mixed population of cells that are enhanced in stem and progenitor cells that are uniquely suited to human administration for tissue repair, tissue regeneration, and tissue engineering. These cells are referred to herein as “Tissue Repair Cells” or “TRCs.” The methods and data presented herein demonstrate that TRCs treat critical limb ischemia in patients/subjects who present a vascular occlusion that cannot be resolved by using a standard method of revascularization.

### Tissue Repair Cells (TRCs)

Isolation, purification, characterization, and culture of TRCs is described in WO 2008/054825, the contents of which are incorporated by reference its entirety.

TRCs contain a mixture of cells of hematopoietic, mesenchymal and endothelial cell lineage produced from mononuclear cells. The mononuclear cells are isolated from adult, juvenile, fetal or embryonic tissues. For example, the mononuclear cells are derived from bone marrow, peripheral blood, umbilical cord blood or fetal liver tissue. TRCs are produced from mononuclear cells, for example by an in vitro culture process which results in a unique cell composition having both phenotypic and functional differences compared to the mononuclear cell population that was used as the starting material. Additionally, the TRCs have both high viability and low residual levels of components used during their production.

The viability of the TRC's is at least 50%, 60%, 70%, 75%, 80%, 85%, 90%, 95% or more. Viability is measured by methods known in the art such as trypan blue exclusion. This enhanced viability makes the TRC population more effective in tissue repair, as well as enhances the shelf-life and cryopreservation potential of the final cell product.

By components used during production is meant, but not limited, to culture media components such as horse serum, fetal bovine serum and enzyme solutions for cell harvest. Enzyme solutions include trypsins (animal-derived, microbial-derived, or recombinant), various collagenases, alternative microbial-derived enzymes, dissociation agents, general proteases, or mixtures of these. Removal of these components provide for safe administration of TRC to a subject in need thereof

Preferably, the TRC compositions of the invention contain less than 10, 5, 4, 3, 2, 1 μg/ml bovine serum albumin; less than 5, 4, 3, 2, 1, 0.9, 0.8, 0.7, 0.6, 0.5 μg/ml harvest enzymes (as determined by enzymatic activity) and are substantially free of mycoplasma, endotoxin and microbial (e.g., aerobic, anaerobic and fungi) contamination.

By substantially free of endotoxin is meant that there is less endotoxin per dose of TRCs than is allowed by the FDA for a biologic, which is a total endotoxin of 5 EU/kg body weight per day, which for an average 70 kg person is 350 EU per total dose of TRCs.

By substantially free for mycoplasma and microbial contamination is meant as negative readings for the generally accepted tests know to those skilled in the art. For example, mycoplasma contamination is determined by subculturing a TRC product sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600x for the presence of mycoplasma as by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turbidity) for a 14 day incubation. A cloudy appearance on any day in either medium indicate contamination, with a clear appearance (no growth) testing substantially free of contamination.

The ability of cells within TRCs to form clonogenic colonies compared to BM-MNCs was determined. Both hematopoietic (CFU-GM) and mesenchymal (CFU-F) colonies were monitored (Table 1). As shown in Table 1, while CFU-F were increased 280-fold, CFU-GM were slightly decreased by culturing.

TABLE 1

| | BM MNC Input | TRC Output | |
|---|---|---|---|
| | (E-06) | (E-06) | Fold Exp |
| CFU-GM | 1.7 | 1.1 ± 0.2 | 0.7 ± 0.1 |
| CFU-F | 0.03 | 6.7 ± 1.3 | 280 ± 67 |

Results are the average ± SEM from 8 clinical-scale experiments.

The cells of the TRC composition have been characterized by cell surface marker expression. Table 2 shows the typical phenotype measured by flow cytometry for starting BM MNCs and TRCs. These phenotypic and functional differences highly differentiate TRCs from the mononuclear cell starting compositions.

TABLE 2

| | | BM MNC Input | | TRC Output | | |
|---|---|---|---|---|---|---|
| | | | Total | | Total | Fold |
| Line- | | | (in | | (in | Expan- |
| age | Marker | % | millions) | % | millions) | sion |
| M | CD105/166 | 0.03 | 0.1 | 12 | 16 | 373 |
| H | CD14auto+ | 0.2 | 0.5 | 26 | 36 | 81 |
| M | CD90 | 0.4 | 0.9 | 22 | 28 | 39 |
| H (E) | CXCR4/VEGFR1 | 0.7 | 1.9 | 12 | 9.9 | 21 |
| E | CD144/146 | 0.5 | 1.3 | 2.7 | 3.2 | 6.3 |
| E | VEGFR1 | 7.6 | 22 | 26 | 38 | 2.3 |
| E | VEGFR2 | 12 | 37 | 25 | 37 | 1.3 |
| H | CD14auto− | 11 | 31 | 14 | 17 | 0.9 |
| H | CD11b | 59 | 162 | 64 | 83 | 0.5 |
| H | CD45 | 97 | 269 | 80 | 104 | 0.4 |
| H | CD3 | 24 | 67 | 8.6 | 11 | 0.2 |

M = mesenchymal lineage, H = hematopoietic lineage, E = endothelial lineage. Results are the average of 4 clinical-scale experiments.

Markers for hematopoietic, mesenchymal, and endothelial lineages were examined. Most hematopoietic lineage cells, including CD11b myeloid, CD14auto-monocytes, CD34 progenitor, and CD3 lymphoid, are decreased slightly, while CD14auto+macrophages, are expanded 81-fold. The mesenchymal cells, defined by CD90⁺ and CD105+/166+/45−/14− have expansions up to 373-fold. Cells that may be involved in vascularization, including mature vascular endothelial cells (CD144/146) and CXCR4/VEGFR1+ supportive cells have expansions between 6- to 21-fold.

Although most hematopoietic lineage cells do not expand in these cultures, the final product still contains close to 80% CD45+ hematopoietic cells and approximately 20% ^{CD90+}mesenchymal cells.

TRCs are highly enriched for CD90⁺ cells compared to the mononuclear cell population from which they are derived. The cells in the TRC composition are at least 5%, 10%, 25%, 50%, 75%, or more CD90⁺. The remaining cells in the TRC composition are CD45⁺. Preferably, the cells in the TRC composition are about 5-75% viable CD90⁺. In various aspects, at least 5%, 10%, 15% , 20%, 25%, 30%, 40%, 50%, 60% or more of the CD90⁺ are also CD15⁺ (Table 3). In addition, the CD90⁺ are also CD105⁺.

TABLE 3

| | TRC | TRC |
|---|---|---|
| | Run 1 | Run 2 |
| % CD90+ | 29.89 | 18.08 |
| % CD90+ CD15− | 10.87 | 3.18 |
| % CD90+ CD15+ | 19.02 | 14.90 |
| % CD15+ of the CD90s | 63.6 | 82.4 |

In contrast, the CD90⁺ population in bone marrow mononuclear cells (BMMNC) is typically less than 1% with the resultant CD45⁺ cells making up greater than 99% of the nucleated cells in BMMNCs Thus, there is a significant reduction of many of the mature hematopoietic cells in the TRC composition compared to the starting mononuclear cell population (Table 2).

This unique combination of hematopoietic, mesenchymal and endothelial stems cells are not only distinct from mononuclear cells but also other cell compositions currently being used in cell therapy. Table 4 demonstrates the cell surface marker profile of TRC compared to mesenchymal stem cells and adipose derived stem cells. (Deans R J, Moseley A B. 2000. Exp. Hematol. 28: 875-884; Devine S M. 2002. J Cell Biochem Supp 38: 73-79; Katz A J, et al. 2005. Stem Cells. 23:412-423; Gronthos S, et al. 2001. J Cell Physiol 189:54-63; Zuk P A, et al. 2002. Mol Biol Cell. 13: 4279-95.)

For example, mesenchymal stem cells (MSCs) are highly purified for CD90⁺ (greater than 95% CD90⁺), with very low percentage CD45⁺ (if any). Adipose-derived stem cells are more variable but also typically have greater than 95% CD90⁺, with almost no CD45⁺ blood cells as part of the composition. There are also Multi-Potent Adult Progenitor Cells (MAPCs), which are cultured from BMMNCs and result in a pure CD90 population different from MSCs that co-expresses CD49c. Other stem cells being used are highly purified cell types including CD34⁺ cells, AC133⁺ cells, and CD34⁺lin⁻ cells, which by nature have little to no CD90⁺ part of the composition and thus are substantially different from TRCs.

Cell marker analysis have also demonstrated that the TRCs isolated according to the methods of the invention have higher percentages of CD14⁺ auto', CD34⁺ and VEGFR⁺ cells.

TABLE 4

| | | | | Adipose- |
|---|---|---|---|---|
| CD | | | Mesenchymal | Derived Stem |
| Locus | Common Name | TRC | stem cells | Cells |
| CD 34 | — | + | − | ± |
| CD13 | gp150 | + | Na | + |
| CD15 | LewisX, SSEA-1 | + | − | − |
| CD11b | Mac-1 | + | − | ± |
| CD14 | LPS receptor | + | − | − |
| CD235a | glycophorin A | + | Na | Na |
| CD45 | Leukocyte common | + | − | − |
| | antigen | | | |
| CD90 | Thy1 | + | + | + |
| CD105 | Endoglin | + | + | + |
| CD166 | ALCAM | + | + | + |
| CD44 | Hyaluronate receptor | + | + | + |
| CD133 | AC133 | + | − | ± |
| — | vWF | + | Na | Na |
| CD144 | VE-Cadherin | + | − | + |
| CD146 | MUC18 | + | + | Na |
| CD309 | VEGFR2, KDR | + | Na | Na |

Each of the cell types present in a TRC population have varying immunomodulatory properties. Monocytes/macrophages (CD45⁺, CD14⁺) inhibit T cell activation, as well as showing indoleamine 2,3-dioxygenase (IDO) expression by the macrophages. (Munn D. H. and Mellor A. L., Curr Pharm Des., 9:257-264 (2003); Munn D. H., et al. J Exp Med., 189:1363-1372 (1999); Mellor A. L. and Munn D. H., J. Immunol., 170:5809-5813 (2003); Munn D H., et al., J. Immunol., 156:523-532 (1996)). Monocytes and macrophages regulate inflammation and tissue repair. (Duffield J. S., Clin Sci (Lond), 104:27-38 (2003); Gordon, S.; Nat. Rev. Immunol., 3:23-35 (2003); Mosser, D. M., J. Leukoc. Biol., 73:209-212 (2003); Philippidis P., et al., Circ. Res., 94:119-126 (2004). These cells also induce tolerance and transplant immunosuppression. (Fandrich F et al. Hum. Immunol., 63:805-812 (2002)). Regulatory T-cells (CD45⁺ CD4⁺ CD25″) regulate innate inflammatory response after injury. (Murphy T. J., et al., J. Immunol., 174:2957-2963 (2005)). The T-cells are also responsible for maintenance of self tolerance and prevention and suppression of autoimmune disease. (Sakaguchi S. et al., Immunol. Rev., 182:18-32 (2001); Tang Q., et al., J. Exp. Med., 199:1455-1465 (2004)) The T-cells also induce and maintain transplant tolerance (Kingsley C. I., et al. J. Immunol., 168:1080-1086 (2002); Graca L., et al., J. Immunol., 168:5558-5565 (2002)) and inhibit graft versus host disease (Ermann J., et al., Blood, 105:2220-2226 (2005); Hoffmann P., et al., Curr. Top. Microbiol. Immunol., 293:265-285 (2005); Taylor P. A., et al., Blood, 104:3804-3812 (2004). Mesenchymal stem cells (CD45⁺ CD90⁺ CD105⁺) express IDO and inhibit T-cell activation (Meisel R., et al., Blood, 103:4619-4621 (2004); Krampera M., et al., Stem Cells, (2005)) as well as induce anti-inflammatory activity (Aggarwal S. and Pittenger M. F., Blood, 105:1815-1822 (2005)).

TRCs also show increased expression of programmed death ligand 1 (PDL1). Increased expression of PDL1 is associated with production of the anti-inflammatory cytokine IL-10. PDL1 expression is associated with a non-inflammatory state. TRCs have increased PDL1 expression in response to inflammatory induction, showing another aspect of the anti-inflammatory qualities of TRCs.

TRCs, in contrast to BM MNCs also produce at least five distinct cytokines and one regulatory enzyme with potent activity both for wound repair and controlled down-regulation of inflammation Specifically, TRCs produce 1) Interleukin-6 (IL-6), 2) Interleukin-10 (IL-10), 3) vascular endothelial growth factor (VEGF), 4) monocyte chemoattractant protein-1 (MCP-1) and, 5) interleukin-1 receptor antagonist (IL-bra). The characteristics of these five cytokines is summarized in Table 5, below.

TABLE 5
Characteristics of TRC Expressed Cytokines.

| CYTO- | |
|---|---|
| KINE | CHARACTERISTIC |
| IL-1 ra | Decoy receptor for IL-1 down-regulates inflammation. IL-1 |
| | ra and IL-10 are characteristically produced by alternatively |
| | activated macrophages |
| IL-6 | Interleukin-6 (IL-6) is a pleiotropic cytokine with a wide range |
| | of biological activities. This cytokine regulates polarization of |
| | naive CD4⁺ T-cells toward the Th2 phenotype, further promotes |
| | Th2 differentiation by up-regulating NFAT1 expression and |
| | inhibits proinflammatory Thl differentiation by inducing |
| | suppressor of cytokine signaling SOCS1. |
| IL-10 | Produced by cell types mediating anti-inflammatory activities, |
| | Th2 type immunity, immunosuppression and tissue repair. IL-10 |
| | and IL-1ra are characteristically produced by alternatively |
| | activated macrophages. IL-10 also is involved in the induction of |
| | regulatory T-cells. In addition, regulatory T-cells secrete high |
| | levels of IL-10. |
| MCP-1 | MCP-1 inhibits the adoptive transfer of autoimmune disease in |
| | animal models and drives TH2 differentiation indicating an anti |
| | inflammatory property particularly when balanced a against |
| | MIP-1α. |
| VEGF | Angiogenic cytokine with simultaneous immunosuppressive |
| | properties acting at the level of the antigen presenting cell. |

Additional characteristics of TRCs include a failure to spontaneously produce, or very low-level production of certain pivotal mediators known to activate the Thi inflammatory pathway including interleukin-alpha (IL-1α), interleukin-beta (IL-1β) interferon-gamma (IFN-γ) and most notably interleukin-12 (IL-12). Importantly, the TRCs neither produce these latter Thi-type cytokines spontaneously during medium replacement or perfusion cultures nor after intentional induction with known inflammatory stimuli such as bacterial lipopolysaccharide (LPS). TRCs produced low levels of IFN-γ only after T-cell triggering by anti-CD3 mAb. Finally, the TRCs produced by the current methods produce more of the anti-inflammatory cytokines IL-6 and IL-10 as well as less of the inflammatory cytokine IL-12.

Moreover, TRCs are inducible for expression of a key immune regulatory enzyme designated indoleamine-2,-3 dioxygenase (IDO). The TRCs according to the present invention express higher levels of IDO upon induction with interferon-γ. IDO has been demonstrated to down-regulate both nascent and ongoing inflammatory responses in animal models and humans (Meisel R., et al., Blood, 103:4619-4621 (2004); Munn D. H., et al., J. Immunol., 156:523-532 (1996); Munn D. H., et al. J. Exp. Med. 189:1363-1372 (1999); Munn D. H. and Mellor A. L., Curr. Pharm. Des., 9:257-264 (2003); Mellor A. L. and Munn D. H., J. Immunol., 170:5809-5813 (2003)).

As discussed above, TRCs are highly enriched for a population of cells that co-express CD90 and CD15.

CD90 is present on stem and progenitor cells that can differentiate into multiple lineages. These cells are a heterogeneous population of cells that are at different states of differentiation. Cell markers have been identified on stem cells of embryonic or fetal origin that define the differentiation state of the cell. One of these markers, SSEA-1, also referred to as CD15, is found on mouse embryonic stem cells, but is not expressed on human embryonic stem cells. It has however been detected in neural stem cells in both mice and human. CD15 is also not expressed on purified mesenchymal stem cells derived from human bone marrow or adipose tissue (Table 6). Thus, the cell population in TRCs that co-expresses both CD90 and CD15 is a unique cell population and may define a the stem-like state of the CD90 adult-derived cells.

Accordingly, in another aspect of the invention the cell population expressing both CD90 and CD15 may be further enriched. By further enriched is meant that the cell composition contains 5%, 10%, 25%, 50%, 75%, 80%, 85%, 90%, 95%, 96%, 97%, 98% 99% or 100% CD90⁺CD15⁺ cells. TRCs can be further enriched for CD90⁺ CD15⁺ cells by methods known in the art such as positive or negative selection using antibodies direct to cell surface markers. The TRCs that have been further enriched for CD90⁺ CD15⁺ cells are particularly useful in cardiac repair and regeneration.

TABLE 6

| Cell Phenotype | TRC | MSC P0 |
|---|---|---|
| % CD90+ | 23.99 | 98.64 |
| % CD15+ | 39.89 | 0.76 |
| % CD15+/CD90+ | 19.54 | 0.22 |
| N | 2 | 4 |

Methods of Production of TRCs

TRCs are isolated from any mammalian tissue that contains bone marrow mononuclear cells (BM MNC). Suitable sources for BM MNC is peripheral blood, bone marrow, umbilical cord blood or fetal liver. Blood is often used because this tissue is easily obtained. Mammals include for example, a human, a primate, a mouse, a rat, a dog, a cat, a cow, a horse or a pig.

The culture method for generating TRCs begins with the enrichment of BM MNC from the starting material (e.g., tissue) by removing red blood cells and some of the polynucleated cells using a conventional cell fractionation method. For example, cells are fractionated by using a FICOLL® density gradient separation. The volume of starting material needed for culture is typically small, for example, 40 to 50 mL, to provide a sufficient quantity of cells to initiate culture. However, any volume of starting material may be used.

Nucleated cell concentration is then assessed using an automated cell counter, and the enriched fraction of the starting material is inoculated into a biochamber (cell culture container). The number of cells inoculated into the biochamber depends on its volume. TRC cultures which may be used in accordance with the invention are performed at cell densities of from 10⁴ to 10⁹ cells per ml of culture. When a Aastrom Replicell Biochamber is used 2-3×10⁸ total cells are inoculated into a volume of approximately 280 mL.

Prior to inoculation, a biochamber is primed with culture medium. Illustratively, the medium used in accordance with the invention comprises three basic components. The first component is a media component comprised of IMDM, MEM, DMEM, RPMI 1640, Alpha Medium or McCoy's Medium, or an equivalent known culture medium component. The second is a serum component which comprises at least horse serum or human serum and may optionally further comprise fetal calf serum, newborn calf serum, and/or calf serum. Optionally, serum free culture mediums known in the art may be used. The third component is a corticosteroid, such as hydrocortisone, cortisone, dexamethasone, solumedrol, or a combination of these, preferably hydrocortisone. The culture medium further comprises B7H3 polypeptides, VSIG4 polypeptides or a combination of both. When the Aastrom Replicell Biochamber is used, the culture medium consists of IMDM, about 10% fetal bovine serum, about 10% horse serum, about 5 μM hydrocortisone, and 4 mM L-Glutamine. The cells and media are then passed through the biochamber at a controlled ramped perfusion schedule during culture process. The cells are cultured for 2, 4, 6, 8, 10, 12, 14, 16 or more days. Preferably, the cells are cultured for less than 12 days. Not to be bound by theory, but it is thought that the addition of B7H3 polypeptides, VSIG4 polypeptides or both will allow for the rapid expansion of TRCs, in particular the CD45⁺, CD31⁺, CD14⁺, and auto⁺ cell population. This rapid expansion will greatly reduce culturing time which is a particular advantage when manufacturing cell suitable for transplantation into humans.

For example, when used with the Aastrom Replicell System Cell Cassette, the cultures are maintained at 37° C. with 5% CO₂ and 20% O₂.

These cultures are typically carried out at a pH which is roughly physiologic, i.e. 6.9 to 7.6. The medium is kept at an oxygen concentration that corresponds to an oxygen-containing atmosphere which contains from 1 to 20 vol. percent oxygen, preferably 3 to 12 vol. percent oxygen. The preferred range of O₂ concentration refers to the concentration of O₂ near the cells, not necessarily at the point of O₂ introduction which may be at the medium surface or through a membrane.

Standard culture schedules call for medium and serum to be exchanged weekly, either as a single exchange performed weekly or a one-half medium and serum exchange performed twice weekly. Preferably, the nutrient medium of the culture is replaced, preferably perfused, either continuously or periodically, at a rate of about 1 ml per ml of culture per about 24 to about 48 hour period, for cells cultured at a density of from 2×10⁶ to 1×10⁷ cells per ml. For cell densities of from 1×10⁴ to 2×10⁶ cells per ml the same medium exchange rate may be used. Thus, for cell densities of about 10⁷ cells per ml, the present medium replacement rate may be expressed as 1 ml of medium per 10⁷ cells per about 24 to about 48 hour period. For cell densities higher than 10⁷ cells per ml, the medium exchange rate may be increased proportionality to achieve a constant medium and serum flux per cell per unit time

A method for culturing bone marrow cells is described in Lundell, et al., “Clinical Scale Expansion of Cryopreserved Small Volume Whole Bone Marrow Aspirates Produces Sufficient Cells for Clinical Use,” J. Hematotherapy (1999) 8:115-127 (which is incorporated herein by reference). Bone marrow (BM) aspirates are diluted in isotonic buffered saline (Diluent 2, Stephens Scientific, Riverdale, N.J.), and nucleated cells are counted using a Coulter ZM cell counter (Coulter Electronics, Hialeah, Fla.). Erythrocytes (non-nucleated) are lysed using a Manual Lyse (Stephens Scientific), and mononuclear cells (MNC) are separated by density gradient centrifugation (Ficoll-Paque® Plus, Pharmacia Biotech, Uppsala, Sweden) (specific gravity 1.077) at 300 g for 20 min at 25° C. BM MNC are washed twice with long-term BM culture medium (LTBMC) which is Iscove's modified Dulbecco's medium (IMDM) supplemented with 4 mM L-glutamine 9GIBCO BRL, Grand Island, N.Y.), 10% fetal bovine serum (FBS), (Bio-Whittaker, Walkersville, M.D.), 10% horse serum (GIBCO BRL), 20 μg/ml vancomycin (Vancocin® HCl, Lilly, Indianapolis, Ind.), 5 μg/ml gentamicin (Fujisawa USA, Inc., Deerfield, Ill.), and 5 μM hydrocortisone (Solu-Cortef®, Upjohn, Kalamazoo, Mich.) before culture.

### Cell Storage

After culturing, the cells are harvested, for example using trypsin, and washed to remove the growth medium. The cells are resuspended in a pharmaceutical grade electrolyte solution, for example Isolyte (B. Braun Medical Inc., Bethlehem, Pa.) supplemented with serum albumin.

Alternatively, the cells are washed in the biochamber prior to harvest using the wash harvest procedure described below. Optionally after harvest the cells are concentrated and cryopreserved in a biocompatible container, such as 250 ml cryocyte freezing containers (Baxter Healthcare Corporation, Irvine, Calif.) using a cryoprotectant stock solution containing 10% DMSO (Cryoserv, Research Industries, Salt Lake City, Utah), 10% HSA (Michigan Department of Public Health, Lansing, Mich.), and 200 μg/ml recombinant human DNAse (Pulmozyme®, Genentech, Inc., South San Francisco, Calif.) to inhibit cell clumping during thawing. The cryocyte freezing container is transferred to a precooled cassette and cryopreserved with rate-controlled freezing (Model 1010, Forma Scientific, Marietta, Ohio). Frozen cells are immediately transferred to a liquid nitrogen freezer (CMS-86, Forma Scientific) and stored in the liquid phase. Preferred volumes for the concentrated cultures range from about 5 mL to about 15 ml. More preferably, the cells are concentrated to a volume of 7.5 mL.

### Post-Culture

When harvested from the biochamber the cells reside in a solution that consists of various dissolved components that were required to support the culture of the cells as well as dissolved components that were produced by the cells during the culture. Many of these components are unsafe or otherwise unsuitable for patient administration. To create cells ready for therapeutic use in humans it is therefore required to separate the dissolved components from the cells by replacing the culture solution with a new solution that has a desired composition, such as a pharmaceutical-grade, injectable, electrolyte solution suitable for storage and human administration of the cells in a cell therapy application.

A significant problem associated with many separation processes is cellular damage caused by mechanical forces applied during these processes, exhibited, for instance, by a reduction in viability and biological function of the cells and an increase in free cellular DNA and debris. Additionally, significant loss of cells can occur due to the inability to both transfer all the cells into the separation apparatus as well as extract all the cells from the apparatus.

Separation strategies are commonly based on the use of either centrifugation or filtration. An example of centrifugal separation is the COBE 2991 Cell Processor (COBE BCT) and an example of a filtration separation is the CYTOMATE® Cell Washer (Baxter Corp) (Table 7). Both are commercially available state-of-the-art automated separation devices that can be used to separate (wash) dissolved culture components from harvested cells. As can be seen in Table 7, these devices result in a significant drop in cell viability, a reduction in the total quantity of cells, and a shift in cell profile due to the preferential loss of the large and fragile CD14⁺ auto⁺ subpopulation of TRCs.

TABLE 7
Performance of 2 different cell separation devices, 3 different studies.

| | COBE 2991 Cell | CYTOMATE ® Cell | CYTOMATE ® Cell |
|---|---|---|---|
| | Processor (n = 3) | Washer (n = 8) | Washer (n = 26) |
| Operating principal | Centrifugation | Filtration | Filtration |
| Study Reference | Aastrom internal protocol | Aastrom new wash | US Fracture Clinical |
| | report #PABI0043 | process development, | Trial, BB-IND #10486 |
| | | report MF#0384 | |
| Average pre-separation | 93% | 93% | 95% |
| cell viability | | | |
| Average post-separation | 83% | 71% | 81% |
| cell viability | | | |
| Average reduction in | 18% | 69% | Not available |
| CD14⁺Auto⁺ frequency | | | |
| Average cell recovery | 73% | 74% | Not available |

These limitations in the art create difficulties in implementing manufacturing and production processes for creating cell populations suitable for human use. It is desirable for the separation process to minimize damage to the cells and thereby result in a cell solution that is depleted of unwanted dissolved components while retaining high viability and biological function with minimal loss of cells. Additionally, it is important to minimize the risk of introducing microbial contaminants that will result in an unsafe final product. Less manipulation and transfer of the cells will inherently reduce this risk.

The invention described in this disclosure overcomes all of these limitations in the current art by implementing a separation process to wash the cells that minimizes exposure of the cells to mechanical forces and minimizes entrapment of cells that cannot be recovered. As a result, damage to cells (e.g. reduced viability or function), loss of cells, and shift in cell profile are all minimized while still effectively separating unwanted dissolved culture components. In a preferred implementation, the separation is performed within the same device that the cells are cultured in which eliminates the added risk of contamination by transfer and separation using another apparatus. The wash process according to the invention is described below.

### Wash Harvest

As opposed to conventional culture processes where cells are removed (harvested) from the biochamber followed by transfer to another apparatus to separate (wash) the cells from culture materials, the wash-harvest technique reverses the order and provides a unique means to complete all separation (wash) steps prior to harvest of the cells from the biochamber.

To separate the culture materials from the cells, a new liquid of desired composition (or gas) may be introduced, preferably at the center of the biochamber and preferably at a predetermined, controlled flow rate. This results in the liquid being displaced and expelled along the perimeter of the biochamber, for example, through apertures, which may be collected in the waste bag.

In some embodiments of the invention, the diameter of the liquid space in the biochamber is about 33 cm, the height of the liquid space is about 0.33 cm and the flow rates of adding rinsing and/or harvesting fluids to the biochamber is about 0.03 to 1.0 volume exchanges (VE) per minute and preferably 0.50 to about 0.75 VE per minute. This substantially corresponds to about 8.4 to about 280 mL/min and preferably 140 to about 210 ml/min. The flow rates and velocities, according to some embodiments, aid in insuring that a majority of the cultured cells are retained in the biochamber and not lost into the waste bag and that an excessively long time period is not required to complete the process. Generally, the quantity of cells in the chamber may range from 10⁴ to 10⁸ cell/mL. For TRCs, the quantity may range from 10⁵ to 10⁶ cells/mL, corresponding to 30 to 300 million total cells for the biochamber dimensions above. Of course, one of skill in the art will understand that cell quantity changes upon a change in the biochamber dimensions

According to some embodiments, in harvesting the cultured cells from the biochamber, the following process may be followed, and is broadly outlined in Table 8, below. The solutions introduced into the biochamber are added into the center of the biochamber. The waste media bag 76 may collect corresponding fluid displaced after each step where a fluid or gas is introduced into the biochamber. Accordingly, after cells are cultured, the biochamber is filled with conditioned culture medium (e.g., IMDM, 10% FBS, 10% Horse Serum, metabolytes secreted by the cells during culture) and includes between about 30 to about 300 million cells. A 0.9% NaCl solution (“rinse solution”) may then be introduced into the biochamber at about 140 to 210 mL per minute until about 1.5 to about 2.0 liters of total volume has been expelled from the biochamber (Step 1).

While a single volume exchange for introduction of a new or different liquid within the biochamber significantly reduces the previous liquid within the biochamber, some amount of the previous liquid will remain. Accordingly, additional volume exchanges of the new/different liquid will significantly deplete the previous liquid.

Optionally, when the cells of interest are adherent cells, such as TRCs, the rinse solution is replaced by harvest solution. A harvest solution is typically an enzyme solution that allows for the detachment of cells adhered to the culture surface. Harvest solutions include for example 0.4% Trypsin/EDTA in 0.9% NaCl that may be introduced into the biochamber at about 140 to 210 mL per minute until about 400 to about 550 ml of total volume has been delivered (Step 2). Thereafter, a predetermined period of time elapses (e.g., 13-17 minutes) to allow enzymatic detachment of cells adhered to the culture surface of the biochamber (Step 3).

Isolyte (B Braun) supplemented with 0.5% HSA may be introduced at about 140 to 210 mL per minute until about 2 to about 3 liters of total volume has been delivered, to displace the enzyme solution (Step 4).

At this point, separation of unwanted solutions (culture medium, enzyme solution) from the cells is substantially complete.

To reduce the volume collected, some of the Isolyte solution is preferably displaced using a gas (e.g., air) which is introduced into the biochamber at a disclosed flow rate (Step 5). This may be used to displace approximately 200 to 250 cc of the present volume of the biochamber.

The biochamber may then be agitated to bring the settled cells into solution (Step 6). This cell suspension may then be drained into the cell harvest bag 70 (or other container) (Step 7). An additional amount of the second solution may be added to the biochamber and a second agitation may occur in order to rinse out any other residual cells (Steps 8 & 9). This final rinse may then be added to the harvest bag 70 (Step 10).

TABLE 8
Wash-harvest Protocol

| Step Number & Name | | Description |
|---|---|---|
| 1 | Rinse out culture media | Use Sodium Chloride to displace the culture medium into |
| | | the waste container. |
| 2 | Add Trypsin solution | Replace Sodium Chloride in culture chamber with the |
| | | Trypsin solution. |
| 3 | Trypsin incubation | Static 15 minute incubation in Trypsin solution. |
| 4 | Rinse out Trypsin solution/Transfer | Add Isolyte with 0.5% HSA to displace the Trypsin |
| | in Pharmaceutically Acceptable Carrier | solution into the waste container. |
| 5 | Concentration/Volume reduction | Displace some of the Isolyte solution with air to reduce |
| | | the final volume (concentration step) |
| 6 | Agitate Biochamber | Rocking motion to dislodge and suspend cells into Isolyte |
| | | solution for collection |
| 7 | Drain into Collection Container | Drain Cells in Isolyte solution into cell collection bag. |
| 8 | Add rinse solution to Biochamber | Add more Isolyte to rinse out residual cells. |
| 9 | Agitate Biochamber | Rocking motion to dislodge and suspend cells into Isolyte |
| | | solution for collection |
| 10 | Drain into Collection Container | Drain the final rinse into the cell collection bag. |

### Therapeutic Methods

Tissue Repair Cells (TRCs) are useful for the treatment of co-option CLI. In certain embodiments of the methods described herein, administration of a TRC composition delays or prevents the progression of no-option CLI over a period of time, which may include the death of the patient (that is not a result of no-option CLI). In other embodiments, administration of a TRC composition improves a symptom of no-option CLI, thereby improving the quality of life for the individual.

### Critical Limb Ischemia (CLI)

Critical Limb Ischemia or CLI is a severe obstruction of the arteries, which decreases blood flow to the extremities (hands, feet and legs). In fact, blood flow is so minimal that when a patient is diagnosed with CLI, he or she presents severe pain that often coincides with the appearance of open wounds that cannot heal (including skin ulcers or sores). The pain caused by CLI is constant and pervades all aspects of life. CLI-associated pain is most noticeable to the patient when he or she is at rest, and, therefore, this pain is also referred to as “rest pain”. Temporary relief from rest pain can be found by moving the limb or walking for a short period of time.

No-option CLI is a form of CLI in which arterial blood flow cannot be restored to the affected limb by using any known or standard method of revascularization. Typically a no-option CLI patient suffers from atherosclerosis or arteriosclerotic vascular disease (AVSD), a condition in which an artery wall thickens as a result of the accumulation of fatty materials such as cholesterol. As a result of underlying arteriosclerotic vascular disease (AVSD), the subject presents a vascular occlusion. If the occlusion is complete, then the individual could be diagnosed with no-option CLI based upon the singular disorder. However, an individual develops no-option CLI for variety of reasons, most of which have a basis in that individual's unique physiology. In many cases, the patient has other underlying medical conditions, such as obesity or diabetes in addition to atherosclerosis. A patient having multiple medical conditions not only presents a vascular occlusion, but may also present additional obstacles to application of standard methods of revascularization, which include either open surgical or percutaneous endovascular procedures. For instance, the location of the occlusion may prevent standard treatment. Alternatively, or in addition, because the patient is obese, diabetic, or aged, the patient may not be otherwise healthy enough for surgery or the subsequent recovery. Because the patient has no acceptable alternative to revascularization, he or she falls within the scope of no-option CLI.

Without treatment, a no-option CLI patient will steadily decline. Current therapies are only sufficient to make the patient more comfortable via pain management and wound care, or to slightly prolong life with amputation. However, a patient who is unfit for revascularization procedures because he or she would not recover from surgery is equally unlikely to recover from an amputation. Consequently, there has been a growing and, prior to the present invention, an unmet need for a treatment for no-option CLI.

According to the methods of the invention, TRCs are delivered to no-option CLI patients using the procedures provided in Examples 1 and 2.

The invention provides a method of treating critical limb ischemia (CLI) in a subject, wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination, thereby improving or preventing the clinical consequence of critical limb ischemia (CLI). The isolated cell composition for tissue repair is also referred to herein as the tissue repair cell (TRC) composition. The formulation of this composition used in the clinical trials described in Examples 1 and 2 is also known as ixmyelocel-T.

the standard method of revascularization is an open surgical procedure or a percutaneous endovascular procedure. The presence of a vascular occlusion that cannot be resolved by using a standard method of revascularization, i.e., the presentation of no-option CLI, may be determined by physical examination, angiographic imaging, color flow duplex ultrasound, or any combination thereof.

The subject may present a vascular occlusion in one or more upper or lower extremities, including any portion thereof. Alternatively, or in addition, the subject may present recurring ischemic rest pain for at least 2 weeks, ulceration, or gangrene with absent pulses in one or more extremities. When a subject presents a vascular occlusion in a lower extremity, the subject may further present recurring ischemic rest pain for at least 2 weeks, ulceration, or gangrene in the foot or toe with absent pedal pulses, and with either a toe systolic pressure of equal to or less than 50 mm Hg or ankle systolic pressure of equal to or less than 70 mm Hg.

Successful treatment of a subject having no-option CLI either avoids a clinical consequence of untreated no-option CLI or achieves a clinical goal following administration of the TRC composition. Moreover, a subject with no-option CLI, who also has an underlying medical condition like morbid obesity, advanced diabetes, or advanced age (with poor general health), may not be able to avoid the more severe consequences of no-option CLI forever, however, they may benefit from these methods by delaying the onset of these events for a sufficient time to experience an increased quality of life. Furthermore, an elderly patient may prolong his or her life by avoiding amputation until morbidity arises from age rather than no-option CLI or an adverse event thereof, thereby, providing an increased quality of life for the interim. Because the subject may already be in poor health, independent of his or her affliction with no-option CLI, the concept of “treating” no-option CLI includes improving mobility, decreasing pain, improving wound healing, decreasing wound size, and delaying tissue loss, amputation, and death. Although the treatment for no-option CLI can be a cure, for instance, in an otherwise healthy individual, the measure of success for treating a subject with no-option CLI in the average subject includes ameliorating an existing symptom or delaying the onset of a worse symptom.

The methods described herein prevent a clinical consequence from occurring when the patient either experiences recovery or when the adverse events associated with no-option CLI are delayed for such a period of time that the patient avoids its occurrence for the duration of his or her life. In an elderly patient, this period of survival may be shorter than in a younger patient, however, in either situation, the endpoint remains recovery or morbidity (by a cause unrelated to no-option CLI). Recovery is defined by either revascularization or sufficient reanimation of the affected limb to be functional. For example, if a patient was using a cane, walker, or wheelchair to aid in walking because of an affected leg that was unable to support his or her body weight, then a functional recovery would include the ability of that individual to support his or her weight, to even to surrender the use of the cane, walker, or wheelchair, depending upon the degree of revascularization. It is contemplated that a subject who regains sufficient function in an affected limb, can sustain motion in this limb and, therefore, through further treatment and physical therapy, permanently avoid amputation.

A clinical consequence of no-option CLI is an adverse event that, without treatment, will inevitably occur as the disease progresses. The term clinical consequence is used to encompass both natural consequences, such as increased pain, wound size, decreased healing, de novo gangrene, and death, and medical consequences such as amputation. Medical consequences are adverse events, compared to what a healthy person might encounter, however, they include necessary interventions to prolong life or improve the quality of life for a patient (e.g. surgery and amputation). Exemplary clinical consequences of no-option critical limb ischemia (CLI) include, but are not limited to, increased rest pain, decreased mobility of a limb (arm or leg), ulceration, increased wound size (doubling of wound size), decreased or impaired wound healing, de novo gangrene, decreased or absent pulse at extremity, tissue loss (tissue necrosis), amputation (for instance, of a digit, such as a finger or toe, which would not constitute a major amputation), major amputation (defined as, for example, an amputation at or above the talus on the leg), or death. Decreased function of an affected limb includes, but is not limited to, decreased range of motion, decreased strength, or decreased endurance for physical exertion of the limb. In certain aspects of this method, the limb is a leg and a decreased function of an affected limb includes decreased walking distance or decreased walking time. Alternatively, or in addition, treatment of the subject having no-option CLI achieves a clinical goal. Exemplary clinical goals include, but are not limited to, decreased pain, increased function of an affected limb, decreased wound size, increased wound healing, delay or prevention of de novo gangrene, delay or prevention of amputation, or increased survival.

When the clinical goal is decreased pain, decreased pain is determined by comparing a demand from the subject for administration of a pain medicine or a dosage of a pain medication from a time period prior to administration of the composition to a demand from the subject for administration of a pain medicine or a dosage of a pain medication from a time point following administration of the composition, wherein a decreased demand or a decreased dosage indicates that the treatment decreased the pain of the subject following administration of the composition.

When the clinical goal is increased function of an affected limb, increased function of an affected limb is determined by comparing a range of motion, a strength, or an endurance measurement for physical exertion of the limb from a time period prior to administration of the composition to a range of motion, a strength, or an endurance measurement for physical exertion of the limb from a time point following administration of the composition, wherein an increased range of motion, increased strength, or increased endurance measurement indicates that the treatment increased the function of the affected limb of the subject following administration of the composition.

When the clinical goal is decreased wound size, decreased wound size is determined by comparing an area, circumference, or depth measurement of a wound from a time period prior to administration of the composition to an area, circumference, or depth measurement of a wound from a time point following administration of the composition, wherein a decreased area, circumference, or depth measurement indicates that the treatment decreased size of a wound following administration of the composition.

When the clinical goal is increased wound healing, increased wound healing is determined by comparing a measurement of active inflammation, angiogenesis, collagen disposition, fibroplasia, granulation tissue formation, epithelialization, contraction, or remodeling of a wound from a time period prior to administration of the composition to a measurement of active inflammation, angiogenesis, collagen disposition, fibroplasias, granulation tissue formation, epithelialization, contraction, or remodeling of a wound from a time point following administration of the composition, wherein an increased measurement of active inflammation, angiogenesis, collagen disposition, fibroplasia, granulation tissue formation, epithelialization, contraction, or remodeling indicates that the treatment increased wound healing following administration of the composition.

When the clinical goal is delay or prevention of de novo gangrene, delay or prevention of de novo gangrene is determined by comparing a measurement of tissue necrosis from a time period prior to administration of the composition to a measurement of tissue necrosis from a time point following administration of the composition, wherein an identical or decreased measurement of tissue necrosis indicates that the treatment delayed or prevented the formation of de novo gangrene following administration of the composition.

When the clinical goal is delay or prevention of amputation, delay or prevention of amputation is determined by comparing the prognosis for amputation in the subject from a time period prior to administration of the composition to the prognosis for either amputation in the subject following administration of the composition, wherein an increase in the time required until amputation or a cancellation of the amputation procedure due to recovery indicates that the treatment delayed or prevented the amputation of the affected limb, respectively.

When the clinical goal is increased survival, increased survival is determined by comparing the prognosis for survival in the subject from a time period prior to administration of the composition to the prognosis for survival in the subject following administration of the composition, wherein an increase in predicted survival time indicates that the treatment increased survival of the subject following administration of the composition.

The TRC composition, also known as ixmyelocel-T, is administered by intramuscular or intravascular injection at one or more sites. Preferably, the composition is administered by intramuscular injection at approximately 20 sites. The TRC composition may be delivered through a wide range of needle sizes, from large 16 gauge needles to very small 30 gauge needles, as well as very long 28 gauge catheters for minimally invasive procedures.

The cells of the composition are derived from mononuclear cells. These mononuclear cells are derived from bone marrow, peripheral blood, umbilical cord blood or fetal liver.

Optionally, the cells of the composition are in formulated or provided in a pharmaceutical-grade electrolyte solution suitable for human administration. The composition is substantially free of horse serum and/or fetal bovine serum.

In certain aspects of the invention, at least 10% of the CD90⁺ cells of the composition co-express CD15. Alternatively, or in addition, the CD45⁺ cells of the composition are CD14⁺, CD34⁺ or VEGFR1⁺.

The total number of viable cells in the composition is between 35 million and 300 million. Preferably, the composition contains an average of between 90-180×10⁶ viable cells. The cells may be suspended in a volume of equal to or less than 15 milliliters, equal to or less than 10 milliliters, or equal to or less than 7.5 milliliters.

Specifically, the invention provides a method of increasing amputation-free survival in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. Optionally, the amputation-free survival is increased in the treated subject when compared to an untreated subject, wherein the untreated subject is also diagnosed with critical limb ischemia (CLI) and also presents a vascular occlusion that cannot be resolved by using a standard method of revascularization. Amputation-free survival is defined as the time of administration of the composition until an amputation is performed, the subject dies, or the combination occurs.

The invention provides a method of preventing major amputation in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. The vascular occlusion may occur in a leg. When the vascular occlusion occurs in a leg, the major amputation is an amputation at or above the talus on the leg. In certain aspects of this method, a major amputation is prevented from the time of administration of the composition until the passage of 1, 2, 3, 4, 5, 6, 7, 8, 9, 10, 15, 20, or 25 years. Alternatively, or in addition, a major amputation is prevented because the extremity is revascularized, as confirmed, for instance, by physical examination, angiographic imaging, color flow duplex ultrasound, or any combination thereof. A subject who experiences revascularization in combination with function of the extremity will avoid major amputation indefinitely, and, therefore, the major amputation has been prevented.

The invention provides a method of delaying the onset of de novo gangrene, tissue loss, amputation, or death in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. Optionally, the onset of de novo gangrene, tissue loss, amputation, or death is delayed in the treated subject when compared to an untreated subject, wherein the untreated subject is also diagnosed with critical limb ischemia (CLI) and also presents a vascular occlusion that cannot be resolved by using a standard method of revascularization. According to this specific method, the term amputation includes both minor and major amputation.

The invention provides a method of increasing survival probability in a subject diagnosed with critical limb ischemia (CLI), wherein the subject presents a vascular occlusion that cannot be resolved by using a standard method of revascularization, including administering to the subject an isolated cell composition for tissue repair including a mixed population of cells of hematopoietic, mesenchymal and endothelial lineage, wherein the viability of the cells is at least 80% and the composition contains: a) about 5-75% viable CD90⁺ cells with the remaining cells in the composition being CD45⁺; b) less than 2 μg/ml of bovine serum albumin; c) less than 1 μg/ml of a enzymatically active harvest reagent; and d) substantially free of mycoplasma, endotoxin, and microbial contamination. Optionally, the survival probability is increased in the treated subject when compared to an untreated subject, wherein the untreated subject is also diagnosed with critical limb ischemia (CLI) and also presents a vascular occlusion that cannot be resolved by using a standard method of revascularization. Survival probability is an alternative method of expressing the time to treatment failure, or the likelihood that the treatment will be successful. The data provided herein demonstrate that individuals with no-option CLI experience a statistically significant benefit from administration of this composition because they survive for a longer time, compared to untreated individuals, before experiencing a no-option CLI-induced adverse event.

In certain embodiments of the methods provided herein, the composition is administered to a subject who presents a vascular occlusion that cannot be resolved by using a standard method of revascularization, in combination with another therapy. For instance, if the subject suffers from an underlying atherosclerosis in the limb undergoing treatment, or in another part of his or her body, the composition is administered in combination with a pharmaceutical agent. Contemplated pharmaceutical agents reduce lipids (lipid or cholesterol reduction therapy), reduce platelet aggregation or platelet attachment to the walls of the vasculature (anti-platelet therapy), or reduce blood pressure (anti-hypertensive therapy). Moreover, the subject of the present methods may have a wound associated with no-option CLI on the treated limb, or on another part of his or her body. Thus, the composition is administered in combination with topical or systemic wound care. Exemplary wound care includes, but is not limited to, pharmaceutical agents to decrease infection (like antibiotics), decrease inflammation, promote healing (antioxidants), and promote vascularization (pro-angiogenic factors); matrices or scaffolds to provide a substrate upon which to grow tissue for the wound; and surgical intervention to removal of dead, damaged, or infected tissue (debridement).

Patients/subjects who develop no-option CLI are often obese, diabetic, and/or elderly. Moreover, these patients experience heart disease at a higher rate than the general population. Thus, the methods provided herein may also be used in combination with treatments for obesity (for instance, drugs including olitstat and the non-prescription version, alli), heart disease (for instance, drugs used to combat high cholesterol or high blood pressure), and diabetes (for example, insulin for type I and weight-loss therapy for type II).

### Pharmaceutical Administration and Dosage Forms

The described TRCs can be administered as a pharmaceutically or physiologically acceptable preparation or composition containing a physiologically acceptable carrier, excipient, or diluent, and administered to the tissues of the recipient organism of interest, including humans and non-human animals. TRC-containing composition can be prepared by resuspending the cells in a suitable liquid or solution such as sterile physiological saline or other physiologically acceptable injectable aqueous liquids. The amounts of the components to be used in such compositions can be routinely determined by those having skill in the art.

The TRCs can be administered by parenteral routes of injection, including subcutaneous, intravenous, intramuscular, and intrasternal. Other modes of administration include, but are not limited to, intrathecal, intracutaneous, and percutaneous. In one embodiment of the present invention, administration of the TRCs can be mediated by endoscopic surgery.

For injectable administration, the composition is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, the TRC can be administered to body tissues, including blood vessel, muscle, skeletal muscle, joints, and limb.

The number of cells in a TRC suspension and the mode of administration may vary depending on the site and condition being treated. As non-limiting examples, in accordance with the present invention, about 35-300×10⁶ TRCs are injected to effect tissue repair. Consistent with the Examples disclosed herein, a skilled practitioner can modulate the amounts and methods of TRC-based treatments according to requirements, limitations, and/or optimizations determined for each case.

In preferred embodiments, the TRC pharmaceutical composition comprises between about 8 and 54% CD90⁺ cells and between about 46 and 92% CD45⁺ cells. The TRC pharmaceutical composition preferably contains between about 35×10⁶ and 300×10⁶ viable nucleated cells and between about 7×10⁶ and 75×10⁶ viable CD90⁺ cells. The TRC pharmaceutical compositional preferably has less than 0.5 EU/ml of endotoxin and no bacterial or fungal growth. In preferred embodiments, a dosage form of TRCs is comprised within 4.7-7.3 mL of pharmaceutically acceptable aqueous carrier. The preferred suspension solution is Multiple Electrolyte Injection Type 1 (USP/EP). Each 100 mL of Multiple Electrolyte Injection Type 1 contains 234 mg of Sodium Chloride, USP (NaCl); 128 mg of Potassium Acetate, USP (C₂H₃KO₂); and 32 mg of Magnesium Acetate Tetrahydrate (Mg(C₂H₃O₂)₂.4H₂O no antimicrobial agents. The pH is adjusted with hydrochloric acid. The pH is 5.5 (4.0 to 8.0). The Multiple Electrolyte Injection Type 1 is preferably supplemented with 0.5% human serum albumin (USP/EP). Preferably, the TRC pharmaceutical composition is stored at 0-12° C., unfrozen.

### Indications and Modes of Delivery for TRCs

TRCs may be manufactured and processed for delivery to patients using the described processes where the final formulation is the TRCs with all culture components substantially removed to the levels deemed safe by the FDA. It is critical for the cells to have a final viability greater than 70%, however the higher the viability of the final cell suspension the more potent and efficacious the final cell dose will be, and the less cellular debris (cell membrane, organelles and free nucleic acid from dead cells), so processes that enhance cell viability while maintaining the substantially low culture and harvest components, while maintaining closed aseptic processing systems are highly desirable.

The invention will be further illustrated in the following non-limiting examples.

### EXAMPLES

### Example 1

### Design and Methods for Trial of Expanded Autologous Bone Marrow Treatment in Patients With No-Option Critical Limb Ischemia (CLI)

To determine the safety and efficacy of intramuscular injection of expanded autologous bone marrow cells (the treatment, also known as “ixmyelocel-T”) in patients with “no-option” critical limb ischemia, a randomized, placebo-controlled, double-blind multi-center phase II clinical trial was launched, which is otherwise known as the RESTORE-CLI trial. Patients who did not receive the ixmyelocel-T treatment were given a placebo control that contained electrolyte solution only.

The primary objective of this study was to determine if ixmyelocel-T can be used safely for restoring peripheral blood flow affected by CLI, using patients with no acceptable alternative to revascularization. The primary endpoints of the study were adverse events (AEs).

The secondary objective of this study was to investigate the efficacy of ixmyelocel-T in treating CLI, and in particular, no-option CLI. The secondary endpoints of the study were time to first occurrence of treatment failure (TTF; major amputation of the treated leg, all-cause mortality, doubling of the total wound surface area from baseline, and de novo gangrene.

Although the study was designed to include 150 subjects, randomization stopped at 86 patients. Seventy-two of these 86 patients received the ixmyelocel-T treatment. The data presented herein include the final analysis of the 12-month data for all 72 patients.

### No-Option CLI

Critical Limb Ischemia (CLI), and, in particular, “no-option” CLI remains a major unmet healthcare need. Up to twenty percent of patients with CLI will die within the first 6 to 12 months; 2-year, 5-year, and 10-year mortality rates are approximately 35%, 70%, and 100%, respectively. As many as 40 to 50% of patients will undergo major limb amputation within 6 to 12 months. The CLI patient population is predominant elderly, and therefore, the ability of this population to successfully rehabilitate and maintain an independent living status following major limb amputation is poor.

CLI is treated using a multi-faced approach. The vascular occlusion may be treated pharmacologically with lipid reduction, anti-platelet and anti-hypertensive therapies. The resultant wounds are treated by standard methods including surgical debridement. Revascularization continues to be the most important method of treatment. Standard methods of revascularization include either open surgical procedures or percutaneous endovascular approaches.

For the most critical of CLI patients, which represent a significant proportion of all CLI patients, effective revascularization using standard methods is not possible, due to location or extent of the disease or associated co-morbidities precluding open surgery. Patients with CLI who are unable to undergo successful revascularization currently have no effective treatment options. There are currently no FDA-approved therapies for CLI. Therapy for this “no-option” CLI patient population is limited to management of the associated co-morbidities with intensive wound care, pain control and eventual amputation of the limb. Thus, the no-option CLI patient represents a population with a serious and life-threatening disease with unmet medical need.

### Methods

The study “Use of Tissue Repair Cells (TRCs-Autologous Bone Marrow Cells) in Patients with Peripheral Arterial Disease to Treat Critical Limb Ischemia” (RESTORE-CLI) is a prospective, randomized, double-blinded, placebo-controlled multi-center study that compares intramuscular injections of expanded autologous bone marrow cells (“Tissue Repair Cells” or TRCs) that are suspended in physiological electrolyte solution with injections of the same electrolyte solution without cells in patients with lower extremity critical limb ischemia (CLI). The study was sponsored by Aastrom Biosciences Inc. in Ann Arbor, Mich. The protocol was reviewed by the Center of Biologics Evaluation and Research (CBER) of the Federal Food and Drug Administration (FDA) and the institutional review boards of the participating centers. All participants provided written voluntary consent. An independent Data Safety Monitoring Board (DSMB) consisting of three expert physicians and one statistician who were not involved in any other aspect of the study monitored the safety of participants in the study according to the DSMB charter specifically developed for RESTORE-CLI.

Eligible patients were men and women 18 to 90 years of age with a diagnosis of CLI of the lower extremities defined as persistent, recurring ischemic rest pain for at least 2 weeks and/or ulceration or gangrene of the foot or toe with absent palpable pedal pulses with toe systolic pressure 50 mm Hg or ankle systolic pressure 70 mm Hg. Patients with flat or barely pulsatile pulse volume recording (PVR) and higher systolic blood pressures could be included based on sponsor review. Patients with infrainguinal occlusive disease without acceptable options for revascularization as determined by the site principles investigator was confirmed by angiographic imaging or color flow duplex ultrasound within 6-months prior to randomization were eligible. Establishment of controlled blood pressure with anti-hypertensive therapy as necessary, adequate anti-platelet and statin therapy was required prior to entry.

Main exclusion criteria were poorly controlled diabetes (defined as, HbA_{1c}>10%); known aortoiliac disease with >50% stenosis; wound with exposed tendon or bone (or a wound severity of greater than Grade 3 on the Wagner Wound Scale); known failed ipsilateral revascularization procedure within 2 weeks prior to randomization (defined as failure to restore adequate circulation, i.e. the procedure did not achieve an increase in ABI of 0.15 or more, substantial improvement in PVR, or clinical improvement); previous amputation of the talus or above in the target limb; infection of the involved extremity (manifested by, for example, fever, purulence, and severe cellulitis); and any active wet gangrenous tissue.

Patients were centrally randomized 2:1 (treatment:control). Visits were scheduled on day minus 14 (bone marrow or sham aspiration), day 0 (injection), days 3 and 7, and months 3, 6, 9 and 12.

Enrollment began at 20 clinical sites in the US in April 2007. The planned study population size was originally up to 150 patients. By November 2009, 33 patients had the opportunity to complete the trial (the 12-month follow-up visit), and were included in a prospectively planned first interim analysis. The first interim analysis was expanded to include 13 additional patients that had completed 6 months of follow-up at that time. Only the set of 32 patients completing 12 months and the set of 14 patients completing 6 months of follow-up by November 2009 were unblinded and included in the interim analysis and reported. Enrollment was subsequently halted and all enrolled patients were followed until completing the 12 month efficacy end-point. The final database lock for the study occurred in May 2011.

### Bone Marrow Aspiration, TRC Production and Intra-Muscular Injection

An independent physician (other than the physician performing TRC injections) aspirated approximately 50 mL bone marrow from the posterior iliac crest. Control patients underwent a sham aspiration that involved the insertion of an aspiration needle at the iliac crest without penetration of the iliac periosteum. The aspirate was shipped overnight to Aastrom Bioscience for TRC production. Patients were dropped from the study if the bone marrow was determined to be unsuitable for ex-vivo processing due to insufficient mononuclear cell number or if the TRC product did not meet production specifications for sterility and number of total and CD90⁺ viable cells.

The TRC product was generated in a single-pass perfusion biochamber over approximately 12 days and then transported to the clinical site in a shipping container designed to maintain hypothermic storage conditions (between 0-12° C)(Dennis et al. Stem Cells. 2007 October; 25(10); 2575-82). TRCs are a mixture of nucleated cells cultured from the patient's bone marrow with high viability. TRCs are primarily composed of two cell phenotypes: mesenchymal stem and progenitor cells defined by the CD90⁺ cell surface marker, and hematopoietic and endothelial stem and progenitor cells, defined by the CD45⁺ cell surface marker. The overall cell viability as measured by membrane integrity by dye exclusion is greater than or equal to 70%. The cells are suspended in a physiological solution of HypoThermosol® (BioLife Solutions) and Isolyte® (B. Braun) supplemented with 0.5% human serum albumin (HSA) in a volume between 5.8 to 8.4 mL. Characteristics of TRCs from patients in the RESTORE-CLI interim analysis are presented in Results.

An average of 136 million total viable TRCs, of which 25 million were CD90⁺′ or electrolyte (control) solution was injected into 20 sites in the ischemic lower extremity. Injection sites were mapped by marking four circumferential linear bands around the lower third of thigh, the greatest diameter of the patient's calf, and at one location proximal and one distal to greatest calf diameter; 5 injections of 0.5 mL were given along each band, at least 2.0 cm apart and 0.5 inches into the muscle, to include all muscle groups. An alteration to the injection procedure occurred during the study. After this change, four (4) injections per linear band were administered with the remaining four (4) injections administered on the dorsal or planter surfaces of the foot into the muscle groups

### Study Endpoints: Safety Evaluations

Safety was assessed continually throughout the study via direct evaluation (including physical examination, vital signs measurement and laboratory values) and by spontaneous reporting by the patient during study visits or telephone contacts. The DSMB reviewed safety data on a routine basis.

The primary endpoint of the study was safety, which included adverse events, aspiration site assessment, injection toxicities, and injection site assessments. Amputation rates and wound healing, while also safety endpoints, are described below under Efficacy Evaluations.

Analyses were conducted on the Safety Population, defined as all patients who were randomized and aspirated, regardless if they received their randomized treatment. The primary safety endpoint includes adverse events. AE collection begins after the patient signs the informed consent document and lasts until the 12-month follow-up visit. However, only aspiration-emergent and treatment-emergent AEs are summarized. AEs were reported by intensity and relationship to study drug, and were summarized by number (N) and percent (%) of patients who experience an event by preferred term (PT). For completeness, the AE listing contains AEs experienced by all enrolled (signed informed consent) patients.

### Study Endpoints: Efficacy Evaluations

The efficacy of TRC treatment was assessed by secondary endpoints. Principal efficacy measures included time to first occurrence of treatment failure, amputation-free survival, incidence of major amputation, and wound healing. Study investigators made amputation decisions independently based on their clinical judgment.

The composite treatment failure endpoint was comprised of the following events: major amputation on the treated/injected limb, death, doubling of wound total surface area from baseline (day 0) or occurrence of de novo gangrene. Major amputation was defined as amputation at or above the talus on the limb receiving injections. For a given patient, the time to first occurrence of treatment failure was defined as the earliest day at which any one of the treatment failure events occurred. For patients who did not experience any of the treatment failure events, their last day in the study was used to calculate event-free duration.

The duration of amputation-free survival was defined as the first day on which a major amputation or death was reported. For patients who did not experience a major amputation or death, their last day in the study was used to determine the event-free duration.

Wound healing was evaluated according to 3 separate assessments: severity using the Wagner Wound Scale, complete wound healing and total surface area of wounds. Wagner classification is useful to measure wound depth where as total surface area sis useful to measure changes in more superficial Wagner 1 and 2 classification wounds. Only wounds present at entry into the study were evaluated by the Wagner Wound Scale and for complete wound healing. The total wound surface area was calculated as the sum of the surface area of each individual wound. For a wound that appeared after the baseline evaluation, the baseline surface area for that wound was defined as zero.

12-months: Analyses were conducted on the Efficacy Population, defined as all randomized patients who received treatment. The efficacy endpoints included TTF (time to treatment failure) and AFS (amputation free survival). TTF is defined as the number of days from injection (Study Day 0) to the earliest study day on which any one of the treatment failure events occurred. AFS is defined as the number of days from injection (Study Day 0) to the first study day on which a major amputation or death was reported. A major amputation is defined as an amputation at or above the talus on the treated leg. TTF and AFS were each assessed using Kaplan-Meier (KM) curves, with the p-value from the log-rank test also provided. In addition, Cox proportional hazards (PH) analyses were performed, to obtain an estimate of the treatment effect. For each of the efficacy endpoints, the hazards ratio (HR) and its 95% confidence interval (CI) from the Cox PH analysis were provided in order to describe the size of the treatment effect of ixmyelocel-T.

### Statistical Analysis

As a phase II trial the primary purpose of the trial was exploratory. The planned sample size was based on assuming 100 TRC patients and 50 Control patients and a Control composite primary event rate of 65% at 6 months with alpha =0.05, 2-sided, then there would be over 80% power to detect a 30% treatment effect (a TRC event rate of 39%).

For the first interim analysis, safety and efficacy data were summarized for the randomized and treated patient populations at 6 and 12 months post treatment. The 6- and 12-month populations were defined as all study participants with the opportunity to complete 6 or 12 months of follow-up, respectively, as of November 2009. For the final study database analyses, safety and efficacy data were summarized through 12 months post-treatment.

Amputation free-survival and time to first occurrence of treatment failure were both summarized using Kaplan-Meier plots by treatment group; the p-value from the log-rank test was provided for descriptive purposes. Major amputation rates at 6 and 12 months were analyzed using Fisher's exact test.

The last-observation-carried-forward (LOCF) method was used for wounds removed due to an amputation at or above the wound location, for total wound surface area and Wagner Wound Scale category.

Measurements of wound severity were based on the Wagner Wound Scale categories. Statistical evaluations were based on the most severe wound (highest Wagner score) present at day 0. The number and percent of patients in each Wagner scale category for each time point as well as the number and percent of patients with improving wounds, based on reduction of Wagner score from baseline. Fisher's exact test was used to analyze differences in the proportion of patients experiencing wound improvement between groups.

Complete wound healing was summarized as the number and percent of patients with wounds present at baseline whose wounds were healed at a given time point (wound size of 0 cm and Wagner score of 0 for each wound). Total wound surface area and change from baseline were summarized by descriptive statistics.

### Example 2

### Results of Trial of Expanded Autologous Bone Marrow Treatment in Patients With No-Option Critical Limb Ischemia (CLI) Patient Enrollment and Characteristics

The disposition of the 46 patients who were included in the first interim analysis in the 6-month population is shown in Tables 9A, 9B, 10A and 10B. There were 7 treatment group withdrawals due to withdrawal of consent (1 patient), death (1 patient), not returning to clinic for mandated assessments (3 patients), loss to follow-up (1 patient), and amputation of the injected leg (1 patient; this was not a protocol allowable reason for withdrawal). In the control group the 1 withdrawal was due to death. All patients who withdrew were included in all efficacy analyses. Five of the seven withdrawals in the treatment group occurred after the 6 month time point. At this time point, reasons for patient withdrawal and outcomes are shown in Table 10A. Baseline characteristics for the 72 patients included in the final database analysis are shown in Table 11.

At the time of the first interim analysis, at the 12-Month time point, nine ixmyelocel-T patients discontinued after treatment compared with three Control patients. Ixmyelocel-T patients withdrew for a variety of reasons, as evidenced in Table 10B below. Table 12 gives detailed information for patients who discontinued after treatment. All patients who withdrew, both ixmyelocel-T and Control, were included in all efficacy analyses.

For the final study database, the percentage of diabetic patients was higher in the Control group (63%) than the ixmyelocel-T group (44%) (Table 9B). There is one patient (2%) in the ixmyelocel-T group with a baseline glomerular filtration rate (GFR)≦30; all Control patients have baseline GFR>30. In addition, 29 of 48 (60%) ixmyelocel-T and 16 of 24 (67%) Control patients had wounds at baseline.

TABLE 9A
Patient Disposition for RESTORE-CLI
Interim Analysis - 6-month Population

| Parameter | TRC | Control |
|---|---|---|
| Randomized, n (%) | 32 (100) | 14 (100) |
| Aspirated, n (%) | 32 (100) | 14 (100) |
| Treated, n (%) | 32 (100) | 14 (100) |
| Withdrew after treatment, n (%) | 7 (22) | 1 (7) |
| Reason for withdrawal, n (%) | | |
| Withdrew consent | 1 (3) | 0 (0) |
| Death | 1 (3) | 1 (7) |
| Did not return to clinic | 3 (9) | 0 (0) |
| Loss to follow-up | 1 (3) | 0 (0) |
| Amputation of treated leg | 1 (3) | 0 (0) |
| Completed, n (%) | 16 (50) | 10 (71) |
| Continuing follow-up, n (%) | 9 (28) | 3 (21) |

TRC, tissue repair cells.

TABLE 9B
Patient Disposition for RESTORE-CLI Final Database Analysis

| | | N (%) | | |
|---|---|---|---|---|
| | | ixmyelocel-T | Control | Total |
| | | (N = 58) | (N = 28) | (N = 113) |
| Screened^{a} | | | | 113 (100) |
| Screening Failure^{b} | | | | 27 (24) |
| Reason for Discontinuation^{b} | Ineligible after enrollment | | | 17 (15) |
| | Withdrew consent | | | 2 (2) |
| | Investigator discretion | | | 1 (1) |
| | Adverse Event | | | 6 (5) |
| | Death | | | 1 (1) |
| Randomized^{b} | | 58 (100) | 28 (100) | 86 (76) |
| Discontinued After Randomization^{c} | | 5 (9) | 4 (14) | 9 (10) |
| Reason for Discontinuation^{c} | Ineligible after enrollment | 2 (3) | 2 (7) | 4 (5) |
| | Lack of compliance | 1 (2) | 0 (0) | 1 (1) |
| | Adverse Event | 2 (3) | 2 (7) | 4 (5) |
| Aspirated^{c} | | 53 (91) | 24 (86) | 77 (90) |
| Discontinued After Aspiration^{d} | | 5 (9) | 0 (0) | 5 (6) |
| Reason for Discontinuation^{c} | Inadequate aspiration | 2 (4) | 0 (0) | 2 (3) |
| | Inadequate TRC product | 1 (2) | 0 (0) | 1 (1) |
| | Adverse Event | 1 (2) | 0 (0) | 1 (1) |
| | Other | 1 (2) | 0 (0) | 1 (1) |
| Treated^{d} | | 48 (91) | 24 (100) | 72 (94) |
| Discontinued After Treatment^{e} | | 9 (19) | 3 (13) | 12 (17) |
| Reason for Discontinuation^{e} | Withdrew consent | 1 (2) | 0 (0) | 1 (1) |
| | Investigator discretion | 1 (2) | 0 (0) | 1 (1) |
| | Adverse Event | 1 (2) | 0 (0) | 1 (1) |
| | Death | 3 (6) | 2 (8) | 5 (7) |
| | Other | 3 (6) | 1 (4) | 4 (6) |
| Completed Study^{e} | | 39 (81) | 21 (88) | 60 (83) |

^{a}Patients screened as denominator.
^{b}Patients screened as denominator.
^{c}Patients randomized as denominator.
^{d}Patients aspirated as denominator.
^{e}Patients treated as denominator.

TABLE 10A
Patients Withdrawn at 6-Months: Time in Study, Reason
for Withdrawal, and Contribution to Efficacy Data

| | Study Day of | Reason for | |
|---|---|---|---|
| Treatment | Withdrawal | Withdrawal* | Additional information |
| TRC | 63 | Other: Amputation | Below knee above talus amputation on |
| | | of injected leg | treated side on Day 31. No wound data |
| | | | reported or other events contributing to |
| | | | treatment failure composite. |
| TRC | 407; Last relevant | Investigator | No amputations reported. Wound data |
| | data (labs) reported | discretion: Missed | reported through Month 9. No other |
| | at Month 9/Day 259 | Visit 9; unable to | events contributing to treatment failure |
| | | return | composite. |
| TRC | 210 | Other: Lost to | Above knee amputation on treated side |
| | | follow up; certified | on Day 101. Wound data reported |
| | | letter sent | through Day 7. No other events |
| | | | contributing to treatment failure |
| | | | composite. |
| TRC | 197; Last relevant | Other: Patient did | No amputations were reported. No |
| | data (labs) reported | not return to clinic; | wound data reported. No events |
| | at Month 3/Day 97. | called 3 times; | contributing to treatment failure |
| | | certified letter sent | composite. |
| TRC | 207 | Withdrew consent | Above knee amputation on treated side |
| | | | on Day 32. Wound data reported |
| | | | through Day 7. No other events |
| | | | contributing to treatment failure |
| | | | composite. |
| TRC | 132 | Death | Adverse event of Cardiac Failure |
| | | | Congestive began on Day 114 and |
| | | | ended in death on Day 132. No |
| | | | amputations were reported. Wound data |
| | | | reported through Month 3. Death was |
| | | | event contributing to treatment failure |
| | | | composite. |
| TRC | 380; Last relevant | Other: Patient did | Wound data reported through Month 9. |
| | data (labs) reported | not return for Visit 9 | No events contributing to treatment |
| | at Month 9/Day 254 | | failure composite. |
| Control | 37 | Death | Adverse event of Hypovolemic Shock |
| | | | began on Day 37 and ended in death on |
| | | | the same day. Wound data reported at |
| | | | baseline only. |

TABLE 10B
Summary of Reasons for Discontinuation at 12-Months

N (%)

| | | ixmyelocel-T | Control |
|---|---|---|---|
| | | (N = 48) | (N = 24) |
| Discontinued After Treatment | | 9 (19) | 3 (13) |
| Reason: | Withdrew consent | 1 (2) | 0 (0) |
| | Investigator discretion | 1 (2) | 0 (0) |
| | Adverse Event | 1 (2) | 0 (0) |
| | Death | 3 (6) | 2 (8) |
| | Other | 3 (6) | 1 (4) |

TABLE 11
Patient Demographics for RESTORE-CLI - Final Study Database

| | | ixmyelocel-T | | Control | | Total | |
|---|---|---|---|---|---|---|---|
| | | (N = 48) | | (N = 24) | | (N = 72) | |
| Gender n(%) | Male | 34 | (71) | 14 | (58) | 48 | (67) |
| | Female | 14 | (29) | 10 | (42) | 24 | (33) |
| Age | Mean (SD) | 69.2 | (13.2) | 67.3 | (11.6) | 68.6 | (12.6) |
| | Median (Min, Max) | 73 | (34, 90) | 70 | (40, 85) | 72 | (34, 90) |
| Race/Origin n(%) | White | 40 | (83) | 22 | (92) | 62 | (86) |
| | Asian | 1 | (2) | 0 | (0) | 1 | (1) |
| | Hispanic or Latino | 3 | (6) | 0 | (0) | 3 | (4) |
| | Black or African American | 4 | (8) | 2 | (8) | 6 | (8) |
| | American Indian | 0 | (0) | 0 | (0) | 0 | (0) |
| Smoking Status n(%) | Never Smoked | 8 | (17) | 4 | (17) | 12 | (17) |
| | Current Smoker | 8 | (17) | 9 | (38) | 17 | (24) |
| | Past Smoker | 32 | (67) | 11 | (46) | 43 | (60) |
| Alcohol Consumption n(%) | Unknown | 1 | (2) | 0 | (0) | 1 | (1) |
| | Never | 15 | (31) | 9 | (38) | 24 | (33) |
| | Current | 21 | (44) | 7 | (29) | 28 | (39) |
| | Past | 11 | (23) | 8 | (33) | 19 | (26) |
| Baseline BMI | Mean (SD) | 26.9 | (4.9) | 28.3 | (5.9) | 27.3 | (5.2) |
| | Median (Min, Max) | 27 | (14, 38) | 28 | (19, 40) | 27 | (14, 40) |
| Baseline Creatinine (mg/dL) | Mean (SD) | 1.19 | (0.47) | 1.10 | (0.33) | 1.16 | (0.43) |
| | Median (Min, Max) | 1.1 | (0.5, 2.8) | 1.2 | (0.5, 1.6) | 1.1 | (0.5, 2.8) |
| Patients with Prior Amputation Below Talus of | | 8 | (17) | 2 | (8) | 10 | (14) |
| Treated Limb n(%) | | | | | | | |
| Patients with Known Diabetes n(%) | | 21 | (44) | 15 | (63) | 36 | (50) |
| Patients Known on Dialysis n(%) | | 0 | (0) | 0 | (0) | 0 | (0) |
| Baseline GFR n(%) | <=30 | 1 | (2) | 0 | (0) | 1 | (1) |
| | >30 | 47 | (98) | 24 | (100) | 71 | (99) |

TABLE 12
Patients Discontinued After Treatment (12-month): Time in Study, Reason for Withdrawal and Contribution to Efficacy Data

| | | Study Day of | Reason for | |
|---|---|---|---|---|
| Patient ID | Treatment | Withdrawal | Withdrawal | Treatment Failure Information |
| 101022001 | ixmyelocel-T | 63 | Adverse Event | AEs of “Increase in rest pain,” and “New wound left foot” |
| | | | | began on Day 27 and ended on Day 31, with a “Below knee |
| | | | | above talus” amputation on treated side. No wound data |
| | | | | reported. No other events, besides major amputation on |
| | | | | treated side on Day 31, contributing to treatment failure |
| | | | | composite. |
| 103024001 | ixmyelocel-T | 407; Last data | Investigator | No amputations reported. Wound data reported through |
| | | reported at Month | discretion: Missed | Month 9. No events contributing to treatment failure |
| | | 9/Day 259 | Visit 9; unable to | composite. |
| | | | return | |
| 105027010 | Control | 258 | Death | Adverse event of “Worsening cardiac disease” began on Day |
| | | | | 233 and ended in death on Day 258. No amputations |
| | | | | reported. Wound data through Month 9. Gangrene was first |
| | | | | event contributing to treatment failure composite, on Day 17; |
| | | | | two more reports of gangrene, on Day 23 and on Day 168. |
| 106029003 | ixmyelocel-T | 210 | Other: Lost to follow | “Above knee” amputation on treated side on Day 101. |
| | | | up; certified letter sent | Wound data reported through Day 7. Gangrene was first |
| | | | | event contributing to treatment failure composite, on Day 97; |
| | | | | amputation on Day 101 was second event of treatment |
| | | | | failure composite.. |
| 107030001 | Control | 359 | Other: Unable to | No amputations reported. No baseline wound data reported. |
| | | | complete Visit 9 | No events contributing to treatment failure composite. |
| 110034001 | ixmyelocel-T | 197 | Other: Patient did not | No amputations were reported. No wound data reported. |
| | | | return to clinic | No events contributing to treatment failure composite. |
| 111035006 | ixmyelocel-T | 148 | Death | Adverse event of “Intertrochanteric hip fracture rt |
| | | | | w/deformity” began on Day 139 and ended in death on Day |
| | | | | 148. No amputations reported. Wound data through Month |
| | | | | 3. Death was event contributing to treatment failure |
| | | | | composite. |
| 112036005 | ixmyelocel-T | 207 | Withdrew consent | “Above knee” amputation on treated side on Day 32. Wound |
| | | | | data reported through Day 7. Gangrene was first event |
| | | | | contributing to treatment failure composite, on Day 19; |
| | | | | amputation on Day 32 was second event of treatment failure |
| | | | | composite. |
| 113038001 | ixmyelocel-T | 132 | Death | Adverse event of “Worsening CHF—progressive weakness, |
| | | | | shortness of breath, cough x1 month” began on Day 114 and |
| | | | | ended in death on Day 132. No amputations were reported. |
| | | | | Wound data reported through Month 3. Death was event |
| | | | | contributing to treatment failure composite. |
| 114039001 | ixmyelocel-T | 380; Last data | Other: Patient did not | No amputations reported. Wound data reported through |
| | | reported at Month | return for Visit 9 | Month 9. No events contributing to treatment failure |
| | | 9/Day 254 | | composite. |
| 121049002 | Control | 37 | Death | Adverse event of “Hypovolemic shock” began on Day 37 |
| | | | | and ended in death on the same day. No amputations |
| | | | | reported. Wound data reported at baseline only. Death was |
| | | | | event contributing to treatment failure composite. |
| 121049011 | ixmyelocel-T | 333 | Death | Adverse event of “Worsening renal function” began on Day |
| | | | | 298 and ended in death on Day 333. “Above knee” |
| | | | | amputation on treated side on Day 325. No baseline wound |
| | | | | data reported. Gangrene was first treatment failure event, on |
| | | | | Day 318; major amputation on treated side was second |
| | | | | event, and death was the final treatment failure event. |

Appendix 16.2.1.1; Appendix 16.2.6.5; Appendix 16.2.7.1; Appendix 16.2.7.3; Appendix 16.2.13

### Analysis of Bone Marrow Aspirate and TRC Phenotype

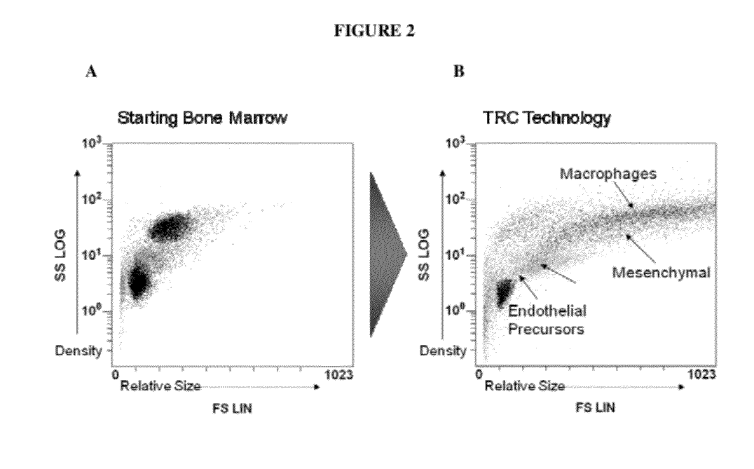

The cell surface phenotype of cells from both the bone marrow aspirates and the TRC product was assessed by flow cytometry in 19 of the patients that received TRCs. The results, presented in FIG. 3, are consistent with established phenotypic differences between unprocessed bone marrow mononuclear cells and culture expanded TRCs (Dennis et al. Stem Cells. 2007 October; 25(10): 2575-82). The total number of cells was decreased by more than half primarily due loss of non-proliferative hematopoietic cells, including mature lymphocytes and granulocytes, which is reflected in the marked decrease in the number CD45⁺ cells. In contrast, the CD90⁺ mesenchymal cell population was expanded about 25-fold from 1 to 25 million cells. Monocytes, as defined by CD14⁺ expression, were expanded by approximately two fold. The specific population of autofluorescent CD14⁺ activated macrophages increased 12 fold, from 0.75 to more than 9 million. There was no significant difference between diabetic and non-diabetic subjects in the ratio of TRCs to BM aspirate cells, or in any of the cell subsets included in FIG. 3 by t-test with significance level=0.05 (data not shown).

### Safety Outcomes

A summary of overall adverse events (AEs) is shown in Table 13. Nearly all patients reported AEs; the proportion of AEs in TRC-treated and control groups was consistent with the 32 to 14 patient randomization. The percentage of patients with serious adverse events (SAEs) was similar between the groups: 44% in TRC-treated and 57% in control patients. There was one death each in the TRC-treatment and in the control groups; neither was considered related to treatment. AEs reported by a total of 4 or more patients in the 6-month population are listed in Table 14. Bone marrow aspiration and injection site toxicities were minimal.

Most severe AEs were determined by investigators to be “unrelated” or “unlikely related” to treatment, but rather to the underlying disease. Two events in treated patients were considered “possibly related” to TRC treatment. One patient experienced moderate cellulitis in the treated limb after bone marrow aspiration but prior to TRC injections. The second patient had a severe localized infection of the first toe of the treated limb recorded on study day 34 that resolved by study day 63. The first ‘cellulitis’ SAE has been down-graded to not related at final analysis. The second SAE of severe localized infection was also updated at final analysis to ‘wound sepsis’ with a start date on day 34 and end date 113. Causality remains the same.

Table 15 provides an overall summary of safety for the Safety Population (aspirated patients) at the end of the study. There were 4 deaths in the ixmyelocel-T group and 2 deaths in the Control group, none considered related to treatment. In the ixmyelocel-T group, three of the patients who died were still participating in the study at the time of their death (one patient died on Study Day 148 related to a hip fracture; one patient died on Study Day 132 related to congestive cardiac failure; and one patient died on Study Day 333 related to renal impairment). The final ixmyelocel-T patient who died had completed the study, but later died of glioblastoma on post-Study Day 498. In the Control group, both patients who died were still participating in the study at the time of their death (one patient died of hypovolemic shock on Study Day 37; and one patient died of cardiac disorder on Study Day 258).

There was one patient in the ixmyelocel-T group and one patient in the Control group that experienced revascularization procedures on the treated limb during the study.

TABLE 13
Overview of Safety at interim - 6-month Population

Parameter

| | TRC | Control |
|---|---|---|
| | N = 32 | N = 14 |
| Patients reporting adverse events, n (%) | 30 (94) | 14 (100) |
| Number of adverse events | 154 | 64 |
| Patients with serious adverse events, n (%) | 14 (44) | 8 (57) |
| Number of serious adverse events | 21 | 15 |
| Number of deaths | 1 | 1 |
| Withdrawals due to adverse events (not | 0 (0) | 0 (0) |
| including deaths), n (%) | | |

TRC, tissue repair cells.

TABLE 14
Most Frequently Reported Adverse Events
at interim - 6-month Population

Preferred Term, n (%)

| | TRC | Control |
|---|---|---|
| | N = 32 | N = 14 |
| Any adverse event | 30 (94) | 14 (100) |
| Pain in extremity | 13 (41) | 3 (21) |
| Skin ulcer | 8 (25) | 1 (7) |
| Nausea | 5 (16) | 3 (21) |
| Gangrene | 4 (13) | 3 (21) |
| Cellulitis | 2 (6) | 4 (29) |
| Diarrhea | 4 (13) | 1 (7) |
| Procedural pain | 4 (13) | 1 (7) |
| Localized infection | 2 (6) | 2 (14) |

TRC, tissue repair cells.

TABLE 15
Final Summary of Safety (12-Months)

Safety Parameter

| | ixmyelocel-T | Control |
|---|---|---|
| | (N = 53) | (N = 24) |
| N (%) with AE | 47 (89) | 23 (96) |
| N (%) Serious AE | 23 (43) | 12 (50) |
| N (%) withdrawal due to AE | 2 (4) | 0 (0) |
| N (%) Deaths * | 3 (6) | 2 (8) |

* An additional ixmyelocel-T patient died~100 days after completing study.

### Efficacy Outcomes

All interim analyses were based on the 6-month or 12-month populations, respectively. Final efficacy analyses included all patients randomized, aspirated, and treated.

### Time to Treatment Failure

Treatment failure was defined as a composite endpoint of major amputation, death, doubling of wound size from baseline or de novo occurrence of gangrene.

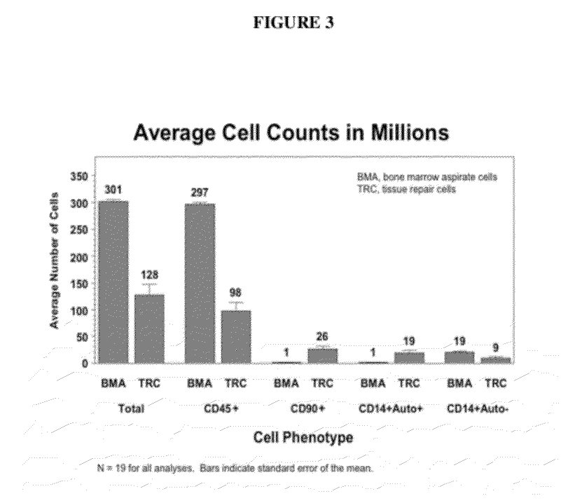

The KM curve from the final analysis in FIG. 4 shows that TTF was statistically significantly longer for ixmyelocel-T patients compared to Control patients (p=0.0032; log-rank test). The Cox PH analysis gave a treatment HR=0.38, 95% CI=(0.20, 0.74), conveying a statistically significant reduction in the risk of treatment failure in the ixmyelocel-T group of approximately 62% (p=0.0047).

Differentiation between the two groups appeared within the first 100 days of follow-up and was maintained through the remainder of the study in the interim analysis. In the 6-month population, 11 of 14 control patients (79%) and 13 of 42 TRC-treated patients (41%) failed treatment by this definition (Fisher's exact test, P=0.026). The etiology of the first occurrence of treatment failure, as well as the total number of patients experiencing each type of failure, is listed in Table 16.

TABLE 16
Occurrence of composite treatment failure endpoint (6-month endpoint)

| | Patient treatment group | | | |
|---|---|---|---|---|
| | TRC (N = 32) | | Control (N = 14) | |
| | Number that failed treatment | | | |
| | 13 (41%) | | 11 (79%) | |
| | Composite endpoint component N(%) | | | |
| | Patients experiencing | Total patients | Patients experiencing | Total patients |
| | as first event | experiencing event | as first event | experiencing event |
| Major amputation | 6 (19%) | 6 (19%) | 4 (29%) | 6 (43%) |
| Death | 1 (3%) | 1 (3%) | 1 (7%) | 1 (7%) |
| Doubling in wound size | 4 (13%) | 6 (19%) | 4 (29%) | 6 (43%) |
| De novo gangrene | 2 (6%) | 4 (13%) | 2 (14%) | 2 (14%) |

### Amputation-Free Survival

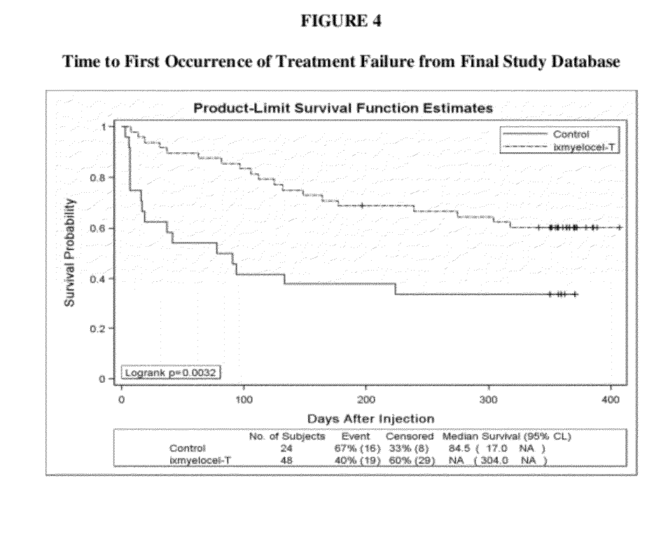

Analysis of the 6-month population in the interim revealed that amputation-free survival (AFS) was significantly longer in the TRC-treated patients compared with control patients (FIG. 5A, logrank test, P=0.038). As with time to treatment failure, differentiation between the distributions appeared within the first 50 days of follow-up and was maintained throughout the study. Median amputation-free survival times for control and TRC-treated patients had not been reached.

At final analysis, the KM curve in FIG. 5B shows that AFS was longer for ixmyelocel-T patients compared to Control patients, but the difference did not reach statistical significance at the 12-month endpoint (p=0.3880; log-rank test). The Cox PH analysis for AFS gave a treatment HR=0.68, 95% CI=(0.28, 1.65), conveying a reduction in the risk of major amputation of the treated limb/death in the ixmyelocel-T group of approximately 32% (p=0.3913).

### TTF and AFS in Subjects With Wounds at Baseline

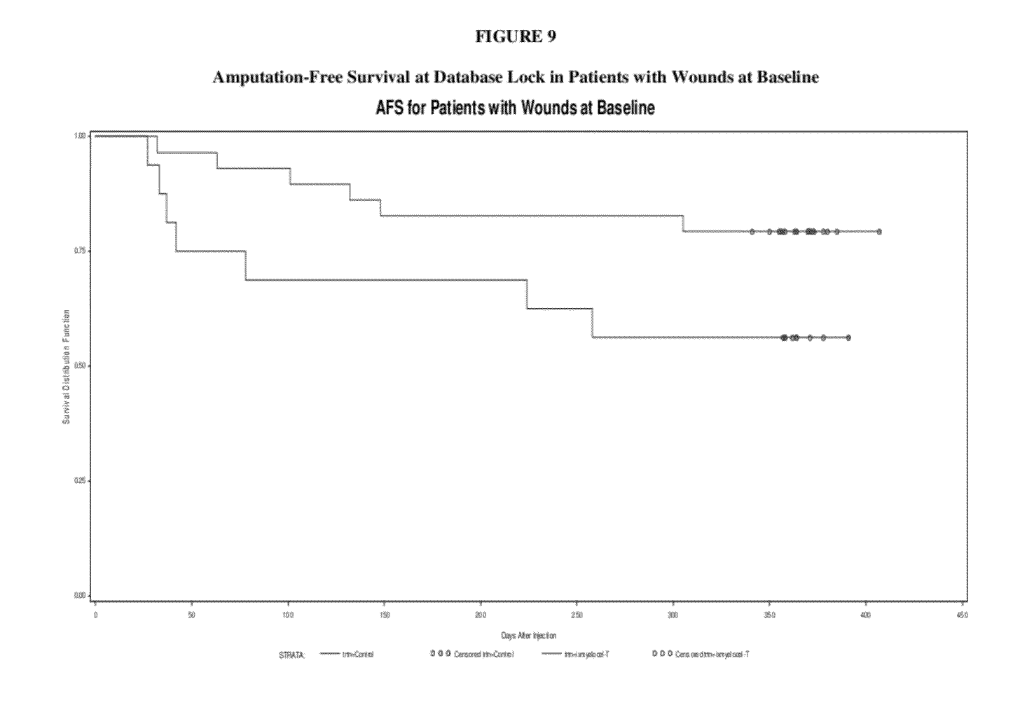

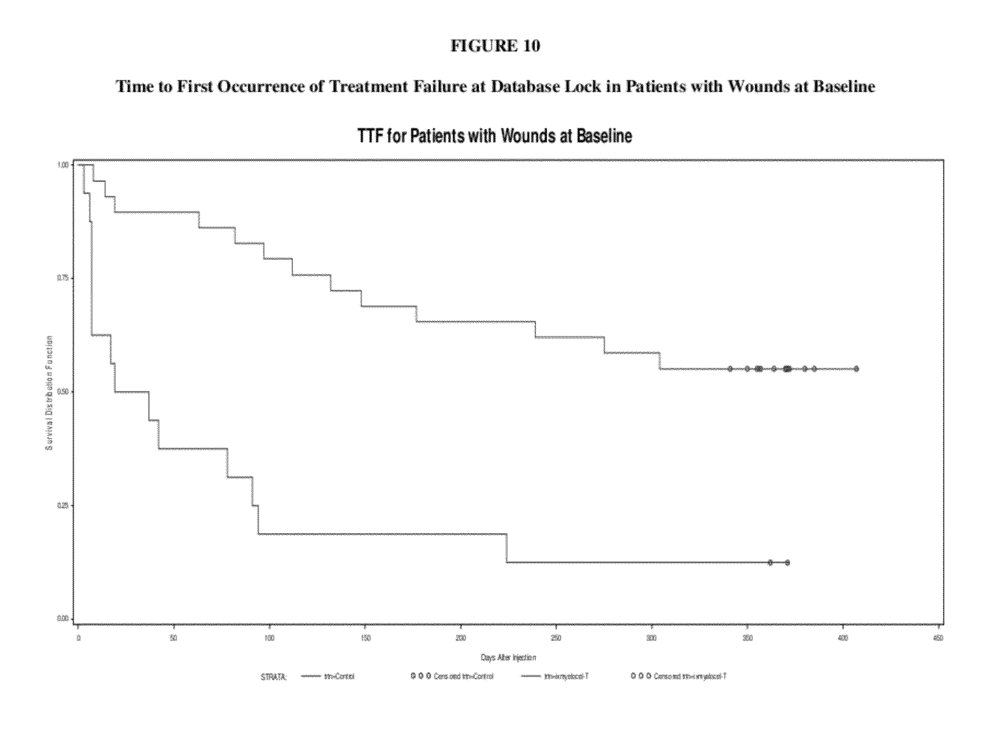

There were a total of 45 patients (29 in the ixmyelocel-T group and 16 in the control group) with wounds at baseline. Both amputation-free survival (AFS) and time to first occurrence of treatment failure (TTF) were analyzed in this subset of patients. FIG. 9 and FIG. 10 give the KM curves for AFS and TTF analysis, respectively.

For AFS, 6 of 29 (20.7%) ixmyelocel-T treated patients with wounds at baseline and 7 of 16 (43.8%) control patients with wounds at baseline had an AFS event (FIG. 9). The analysis indicated that AFS was marginally statistically significantly longer in these ixmyelocel-T treated patients than in control patients (p=0.0802, logrank test).

For TTF, 13 of 29 (44.8%) ixmyelocel-T patients with wounds at baseline and 14 of 16 (87.5%) control patients with wounds at baseline experienced a TTF event (FIG. 10). The analysis indicated that TTF was statistically significantly longer in ixmyelocel-T treated patients than in controls (p<0.0001, logrank test).

### Major Amputation

For the 6-month population at interim, at Month 6, major amputation occurred in 43% of the control group compared with 19% in the treatment group (P=0.14; Fisher's exact test). There was no difference in above versus below the knee amputation between the groups. Below the knee amputation occurred in 66% of patients requiring major amputation in each group. For the 12-month population, major amputation occurred in 36% of the control group compared with 18% in the treatment group at both Months 6 and 12 (P=0.39; Fisher's exact test).

### Wound Healing

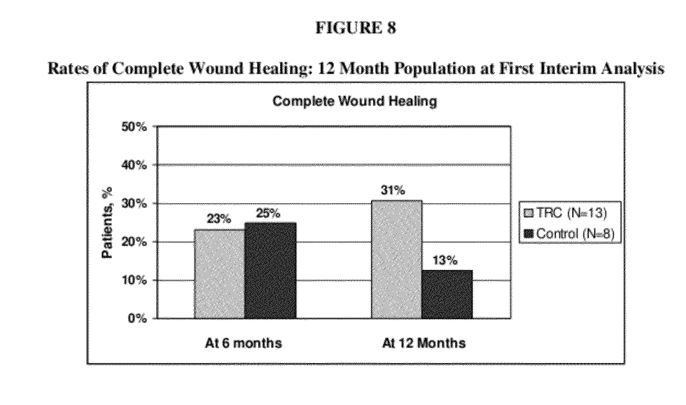

Thirty-three patients had evaluable wounds at baseline that allowed efficacy assessment. Complete wound healing, defined as a Wagner score of 0 and wound size of 0 cm for each wound, was summarized as the percent of patients with wounds present at baseline for whom all wounds have healed at a given time point. Complete wound healing is shown for the 12-month patient population with wounds present at baseline in FIG. 8. For the 12-month population at month 6, there were no differences in complete wound healing between the treatment and control groups. At month 12, complete wound healing was present in a greater percentage of the treatment group (31%) compared to the control group (13%); these differences were not statistically significant. Other measures of wound healing (Wagner Wound Scale, total wound surface area) did not show significant differences between groups.

### Conclusions

RESTORE-CLI is the first placebo-controlled autologous stem cell trial to use expanded bone marrow mononuclear cells (TRCs) to treat no-option CLI patients. It is double-blinded and studies a larger subject population than previously reported cellular therapy studies in CLI. Interim analysis of this phase II trial has demonstrated that TRC therapy is safe and yields potential improvement in efficacy outcomes.

There were no safety issues related to aspiration, injection procedures, or ixmyelocel-T treatment. Furthermore, the frequency and type of AEs reported for both the ixmyelocel-T and Control patients were those to be expected in this seriously ill patient population. Investigators termed the majority of adverse events unrelated to treatment. All details relating to the ixmyelocel-T and Control patients who withdrew from the study were closely examined; the compiled information on these patients did not signal a safety concern.

This Phase 2 trial was not powered to show statistical differences for efficacy endpoints between ixmyelocel-T and Control groups. Efficacy endpoints were analyzed to provide information on the risks and benefits of ixmyelocel-T treatment and further refine the Phase 3 development program. Critically, despite the small number of patients, TTF (major amputation of the treated leg, all-cause mortality, doubling in wound size from baseline, or de novo gangrene) was significantly longer for ixmyelocel-T patients compared to Control patients (p=0.0032; log-rank test) for the Efficacy Population. In addition, there was evidence of longer AFS in the ixmyelocel-T treatment group. Because both non-healing wounds and gangrene are known to be indicators of progression of the disease that lead to amputation, statistical significance on TTF in this small Phase 2 trial gives confidence in reaching statistical significance on AFS in a large Phase 3 trial.

The study was performed primarily to assess safety and tolerability of TRC therapy. As expected in an autologous cell therapy, the administration of TRCs was safe and well tolerated. The number of patients reporting SAEs was similar in the TRC-treated group compared to the control group. RESTORE-CLI was not statistically designed for demonstration of efficacy, which was a secondary aim of the trial. In addition, the interim analysis was performed in only 46 of the 86 subjects at the 6-month endpoint, whereas the final analysis was performed in all 72 ixmyelocel-T treated subjects at the 12-month endpoint. Both the interim and final analyses revealed statistically significant differences in time to first occurrence of treatment failure. The interim analysis found a statistically significant difference in the amputation-free survival between TRC-treated and control subjects, whereas the 12-month report found a clinically-important increase in the amputation-free survival of the treated group. It is anticipated that a phase III trial with enough subjects to power the statistical significant differences between these groups, the amputation-free survival will also be significant at the 12-month endpoint. Wound healing differences, thought not statistically significant, favored TRC-treated patients at the 12-month but not the 6-month time point, consistent with a durable clinical benefit. Similarly, the data collected in this trial support a conclusion that wound healing differences will be statistically significant in a larger study.

Advantages of the cell expansion technique reported here include the need to collect a relatively small amount of bone marrow under local anesthesia. Alternative techniques require harvesting up to 500-600 mL of bone marrow under general anesthesia. Other potential advantages of TRCs are that the expansion process enriches for the cell lineages thought to be important for angiogenesis and neovascularization and may reverse the suppressive effects of chronic medical conditions on bone marrow progenitors that may impair their regenerative function (Lawall H. et al. Thromb Haemost. 2010 Mar 31; 103(4): 696-709).

The analysis provided herein demonstrates that intra-muscular injection of bone marrow-derived TRCs is safe and well tolerated, and provides a significant improvement in amputation-free survival and time to first occurrence of treatment failure when compared to control subjects. These interim results suggest TRCs are a viable option for the treatment of CLI patients without revascularization alternatives.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating critical limb ischemia (CLI) in a subject, wherein the subject presents a vascular occlusion that cannot be resolved by using a standard method of revascularization, comprising administering to the subject an isolated cell composition for tissue repair comprising a mixed population of cells of hematopoietic, mesenchymal and endothelial lineage, wherein the viability of said cells is at least 80% and the composition contains:
a) about 5-75% viable CD90⁺ cells with the remaining cells in said composition being CD45⁺;
b) less than 2 μg/ml of bovine serum albumin;
c) less than 1 μg/ml of a enzymatically active harvest reagent; and
d) substantially free of mycoplasma, endotoxin, and microbial contamination, thereby improving or preventing the clinical consequence of critical limb ischemia (CLI).

2. The method of claim 1, wherein the cells of the composition are derived from mononuclear cells.

3. The method of claim 2, wherein the mononuclear cells are derived from bone marrow, peripheral blood, umbilical cord blood or fetal liver.

4. The method of claim 1, wherein said cells of the composition are in a pharmaceutical-grade electrolyte solution suitable for human administration.

5. The method of claim 1, wherein at least 10% of the CD90⁺ cells of the composition co-express CD15.

6. The method of claim 1, wherein the CD45⁺ cells of the composition are CD14⁺, CD34⁺ or VEGFR1⁺.

7. The method of claim 1, wherein said composition is substantially free of horse serum and/or fetal bovine serum.

8. The method of claim 1, wherein the total number of viable cells in the composition is 35 million to 300 million.

9. The method of claim 8, wherein the cells are in a volume less than 15 milliliters.

10. The method of claim 8, wherein the cells are in a volume less than 10 milliliters.

11. The method of claim 8, wherein the cells are in a volume less than 7.5 milliliters.

12. The method of claim 1, wherein the standard method of revascularization is an open surgical procedure or a percutaneous endovascular procedure.

13. The method of claim 1, wherein the subject presents a vascular occlusion in a lower extremity.

14. The method of claim 1, wherein the subject further presents recurring ischemic rest pain for at least 2 weeks, ulceration, or gangrene with absent pulses in an extremity.

15. The method of claim 13, wherein the subject further presents recurring ischemic rest pain for at least 2 weeks, ulceration, or gangrene in the foot or toe with absent pedal pulses, and with either a toe systolic pressure of equal to or less than 50 mm Hg or ankle systolic pressure of equal to or less than 70 mm Hg.

16. The method of claim 1, wherein the presence of a vascular occlusion that cannot be resolved by using a standard method of revascularization is determined by physical examination, angiographic imaging, color flow duplex ultrasound, or any combination thereof.

17. The method of claim 1 or 13, wherein the composition is administered by intramuscular injection at one or more sites.

18. The method of claim 1 or 13, wherein the composition comprises an average of between 90-180×10⁶ viable cells.

19. The method of claim 1, wherein a clinical consequence of critical limb ischemia (CLI) in the subject is increased pain, decreased function of an affected limb, increased wound size, decreased wound healing, de novo gangrene, amputation, or death.

20. The method of claim 19, wherein decreased function of an affected limb comprises decreased range of motion, decreased strength, or decreased endurance for physical exertion of the limb.

21. The method of claim 19, wherein the limb is a leg and decreased function of an affected limb comprises decreased walking distance or decreased walking time.

22. The method of claim 1, wherein the treatment of the subject achieves a clinical goal.

23. The method of claim 22, wherein the clinical goal is decreased pain, increased function of an affected limb, decreased wound size, increased wound healing, delay or prevention of de novo gangrene, delay or prevention of amputation, or increased survival.

24. The method of claim 23, wherein decreased pain is determined by comparing a demand from the subject for administration of a pain medicine or a dosage of a pain medication from a time period prior to administration of the composition to a demand from the subject for administration of a pain medicine or a dosage of a pain medication from a time point following administration of the composition, wherein a decreased demand or a decreased dosage indicates that the treatment decreased the pain of the subject following administration of the composition.

25. The method of claim 23, wherein increased function of an affected limb is determined by comparing a range of motion, a strength, or an endurance measurement for physical exertion of the limb from a time period prior to administration of the composition to a range of motion, a strength, or an endurance measurement for physical exertion of the limb from a time point following administration of the composition, wherein an increased range of motion, increased strength, or increased endurance measurement indicates that the treatment increased the function of the affected limb of the subject following administration of the composition.

26. The method of claim 23, wherein decreased wound size is determined by comparing an area, circumference, or depth measurement of a wound from a time period prior to administration of the composition to an area, circumference, or depth measurement of a wound from a time point following administration of the composition, wherein a decreased area, circumference, or depth measurement indicates that the treatment decreased size of a wound following administration of the composition.

27. The method of claim 23, wherein increased wound healing is determined by comparing a measurement of active inflammation, angiogenesis, collagen disposition, fibroplasia, granulation tissue formation, epithelialization, contraction, or remodeling of a wound from a time period prior to administration of the composition to a measurement of active inflammation, angiogenesis, collagen disposition, fibroplasias, granulation tissue formation, epithelialization, contraction, or remodeling of a wound from a time point following administration of the composition, wherein an increased measurement of active inflammation, angiogenesis, collagen disposition, fibroplasia, granulation tissue formation, epithelialization, contraction, or remodeling indicates that the treatment increased wound healing following administration of the composition.

28. The method of claim 23, wherein delay or prevention of de novo gangrene is determined by comparing a measurement of tissue necrosis from a time period prior to administration of the composition to a measurement of tissue necrosis from a time point following administration of the composition, wherein an identical or decreased measurement of tissue necrosis indicates that the treatment delayed or prevented the formation of de novo gangrene following administration of the composition.

29. The method of claim 23, wherein delay or prevention of amputation is determined by comparing the prognosis for amputation in the subject from a time period prior to administration of the composition to the prognosis for either amputation in the subject following administration of the composition, wherein an increase in the time required until amputation or a cancellation of the amputation procedure due to recovery indicates that the treatment delayed or prevented the amputation of the affected limb, respectively.

30. The method of claim 23, wherein increased survival is determined by comparing the prognosis for survival in the subject from a time period prior to administration of the composition to the prognosis for survival in the subject following administration of the composition, wherein an increase in predicted survival time indicates that the treatment increased survival of the subject following administration of the composition.

31. A method of increasing amputation-free survival in a subject diagnosed with critical limb ischemia (CLI), wherein the subject presents a vascular occlusion that cannot be resolved by using a standard method of revascularization, comprising administering to the subject an isolated cell composition for tissue repair comprising a mixed population of cells of hematopoietic, mesenchymal and endothelial lineage, wherein the viability of said cells is at least 80% and the composition contains:
a) about 5-75% viable CD90⁺ cells with the remaining cells in said composition being CD45⁺;
b) less than 2 μg/ml of bovine serum albumin;
c) less than 1 μg/ml of a enzymatically active harvest reagent; and
d) substantially free of mycoplasma, endotoxin, and microbial contamination.

32. The method of claim 31, wherein the amputation-free survival is increased in the treated subject when compared to an untreated subject, wherein the untreated subject is also diagnosed with critical limb ischemia (CLI) and also presents a vascular occlusion that cannot be resolved by using a standard method of revascularization.

33. A method of preventing major amputation in a subject diagnosed with critical limb ischemia (CLI), wherein the subject presents a vascular occlusion that cannot be resolved by using a standard method of revascularization, comprising administering to the subject an isolated cell composition for tissue repair comprising a mixed population of cells of hematopoietic, mesenchymal and endothelial lineage, wherein the viability of said cells is at least 80% and the composition contains:
a) about 5-75% viable CD90⁺ cells with the remaining cells in said composition being CD45⁺;
b) less than 2 μg/ml of bovine serum albumin;
c) less than 1 μg/ml of a enzymatically active harvest reagent; and
d) substantially free of mycoplasma, endotoxin, and microbial contamination.

34. The method of claim 33, wherein the vascular occlusion occurs in a leg.

35. The method of claim 34, wherein major amputation is an amputation at or above the talus on the leg.

36. A method of delaying the onset of de novo gangrene, tissue loss, amputation, or death in a subject diagnosed with critical limb ischemia (CLI), wherein the subject presents a vascular occlusion that cannot be resolved by using a standard method of revascularization, comprising administering to the subject an isolated cell composition for tissue repair comprising a mixed population of cells of hematopoietic, mesenchymal and endothelial lineage, wherein the viability of said cells is at least 80% and the composition contains:
a) about 5-75% viable CD90⁺ cells with the remaining cells in said composition being CD45⁺;
b) less than 2 μg/ml of bovine serum albumin;
c) less than 1 μg/ml of a enzymatically active harvest reagent; and
d) substantially free of mycoplasma, endotoxin, and microbial contamination.

37. The method of claim 36, wherein the onset of de novo gangrene, tissue loss, amputation, or death is delayed in the treated subject when compared to an untreated subject, wherein the untreated subject is also diagnosed with critical limb ischemia (CLI) and also presents a vascular occlusion that cannot be resolved by using a standard method of revascularization.

38. A method of increasing survival probability in a subject diagnosed with critical limb ischemia (CLI), wherein the subject presents a vascular occlusion that cannot be resolved by using a standard method of revascularization, comprising administering to the subject an isolated cell composition for tissue repair comprising a mixed population of cells of hematopoietic, mesenchymal and endothelial lineage, wherein the viability of said cells is at least 80% and the composition contains:
a) about 5-75% viable CD90⁺ cells with the remaining cells in said composition being CD45⁺;
b) less than 2 μg/ml of bovine serum albumin;
c) less than 1 μg/ml of a enzymatically active harvest reagent; and
d) substantially free of mycoplasma, endotoxin, and microbial contamination.

39. The method of claim 38, wherein the survival probability is increased in the treated subject when compared to an untreated subject, wherein the untreated subject is also diagnosed with critical limb ischemia (CLI) and also presents a vascular occlusion that cannot be resolved by using a standard method of revascularization.
